SEQUENCE LISTING (123 sequences)

SEQ ID NO: 1 - protein, 62 aa, Unknown Organism
  Description of Unknown Organism Viral peptide
LEDGAYRIKQKGILGYSQIGAGVYKEGTFHTMWHVTRGAVLMHKGKRIEPSWADVKKDLISY

SEQ ID NO: 2 - protein, 62 aa, Langat virus
VKNGVYRIYTPGLLGQRQIGVGYGAKGVLHTMWHVTRGAALLVDGVAVGPYWADVREDVVCY

SEQ ID NO: 3 - protein, 62 aa, Montana myotis leukoencephalitis virus
ITDGVYRIKVSSLFGKKQVGVGVWSEGSFHTMWHVTRGATLRIGDRKLSPEWANITEDLISY

SEQ ID NO: 4 - protein, 62 aa, Powassan virus
IKEGVYRIYAPGLFGYRQIGVGYGTKGVLHTMWHVTRGAALSVEGATSGPYWADVREDVVCY

SEQ ID NO: 5 - protein, 62 aa, Louping ill virus
VKDGVYRIFSPGLFGQRQVGVGYGHKGVLHTMWHVTRGAALSIDDAVAGPYWADVKEDVVCY

SEQ ID NO: 6 - protein, 62 aa, Tick-borne encephalitis virus
VKDGVYRIFSPGLLGQRQVGVGYGSKGVLHTMWHVTRGAALYIDDAVAGPYWADVKEDVVCY

SEQ ID NO: 7 - protein, 62 aa, Omsk hemorrhagic fever virus
VRDGVYRILSPGLLGHRQVGVGFGSKGVLHTMWHVTRGAAIFIDNAVAGPYWADVKEDVVCY

SEQ ID NO: 8 - protein, 62 aa, Modoc virus
LLEGVYRITAASIFGRKQVGIGVLKEGVFHTMWHVTRGSALRVRGRVLAPEWASVEEDLVAY

SEQ ID NO: 9 - protein, 62 aa, Karshi virus
VKDGVYRIFRPGLFGASQIGVGYGSHGVLHTMWHVTRGAAVSINGGAVGPYWADIREDVVCY

SEQ ID NO: 10 - protein, 62 aa, Ilheus virus
LKPGVYRVMTMGILGRYQSGVGVMWDGVFHTMWHVTQGAALRNGEGRLNPTWGSVRDDLISY

SEQ ID NO: 11 - protein, 62 aa, Murray Valley encephalitis virus
TTPGVYRIMARGILGRYQAGVGVMHEGVFHTLWHTTRGAAIMSGEGRLTPYWGNVKEDRVTY

SEQ ID NO: 12 - protein, 62 aa, West Nile virus
TTTGVYRIMTRGLLGSYQAGAGVMVEGVFHTLWHTTKGAALMSGEGRLDPYWGSVKEDRLCY

SEQ ID NO: 13 - protein, 62 aa, Entebbe bat virus
VPDGVYAINQKGLLGSNQRGVGVVKEGVFHTMWHVTRGALLRYEENYITPQWASVKSDLISY

SEQ ID NO: 14 - protein, 62 aa, Unknown Organism
  Description of Unknown Organism Viral peptide
LEDGAYRIKQKLILGYSQIGAGVYKEGTFHTMWHVTRGAVLMHKGKRIEPSWADVKKDLISY

SEQ ID NO: 15 - protein, 62 aa, Zika virus
TTDGVYRVMTRRLLGSTQVGVGVMQEGVFHTMWHVTKGAALRSGEGRLDPYWGDVKQDLVSY

SEQ ID NO: 16 - protein, 62 aa, Kunjin virus; identical to SEQ ID NO: 12

SEQ ID NO: 17 - protein, 62 aa, Japanese encephalitis virus
TTTGVYRIMARGILGTYQAGVGVMYENVFHTLWHTTRGAAIMSGEGKLTPYWGSVKEDRIAY

SEQ ID NO: 18 - protein, 62 aa, Dengue virus type 1
LDDGIYRILQRGLLGRSQVGVGVFQEGVFHTMWHVTRGAVLMYQGKRLEPSWASVKKDLISY

SEQ ID NO: 19 - protein, 62 aa, Kedougou virus
LGDGVYRIMSKKLLGPSQLGVGVMTQGVFHTMWHVTRGCTIEYNGGRLTPYWGSVKEDAISY

SEQ ID NO: 20 - protein, 62 aa, Yokose virus
TPDGVYRIMQKGVFGPQQKGVGVGKSGTFHTMWHVTHGSLLRLNGKLVTPSWANVKKDLIAY

SEQ ID NO: 21 - protein, 62 aa, Dengue virus type 4
LTEGVYRIMQRGLLGKTQVGVGIHMEGVFHTMWHVTRGSVICHETGRLEPSWADVRNDMISY

SEQ ID NO: 22 - protein, 62 aa, Bagaza virus
LSTGVFRIMARGILGKYQAGVGVMEDGVFHTMWHVTNGATINVGDGCLVPYWASVREDLISY

SEQ ID NO: 23 - protein, 62 aa, Rocio virus
MKPGVYRVMTSGVLGSYQSGVGVMYDGVFHTMWHVTQGAALRNGEGRLNPTWGSVRDDLITY

SEQ ID NO: 24 - protein, 62 aa, Alfuy virus
TTPGVYRIMAKGLLGRYQAGVGIMYEGVFHTLWHTTRGAAIMSGEGRLTPPWGNVKEDRITY

SEQ ID NO: 25 - protein, 62 aa, Sepik virus
LEDGVYTINQKNVLGMAQKGVGVVKDGVFHTMWHVTRGAFLLPEGKRLTPAWANVKEDLISY

SEQ ID NO: 26 - protein, 62 aa, Usutu virus
TSPGVYRIMSRYILGTYQAGVGVMYEGVLHTLWHTTRGAAIRSGEGRLTPYWGSVKEDRITY

SEQ ID NO: 27 - protein, 62 aa, West Nile virus; identical to SEQ ID NO: 12

SEQ ID NO: 28 - protein, 62 aa, Dengue virus type 3
LEEGVYRIKQQGIFGKTQVGVGVQKEGVFHTMWHVTRGAVLTHNGKRLEPNWASVKKDLISY

SEQ ID NO: 29 - protein, 62 aa, St. Louis encephalitis virus
TKPGIYRIMTRGILGTFQAGVGVMHEGVFHTMWHATEGAVLRNGEGRLDPYAGDVRNDLISY

SEQ ID NO: 30 - protein, 62 aa, Yellow fever virus
LEDGIYGIFQSTFLGASQRGVGVAQGGVFHTMWHVTRGAFLVRNGKKLIPSWASVKEDLVAY

SEQ ID NO: 31 - protein, 62 aa, Rio Bravo virus
IQDGVYRIMVSSLFGKKQVGVGIWTGNSFHTMWHVTRGAVLNICGRKIVPEWASITEDLISY

SEQ ID NO: 32 - protein, 17 aa, Dengue virus
HRVDLFSPTSYGNGVVY

SEQ ID NO: 33 - protein, 16 aa, Unknown Organism
  Description of Unknown Organism Viral peptide
HRVDLFSPTSYGNGVY

SEQ ID NO: 34 - protein, 16 aa, Dengue virus type 1
HRVDLFKPTSYGNGVY

SEQ ID NO: 35 - protein, 16 aa, Dengue virus type 4
HRVDLFKPTSYGNGVY

SEQ ID NO: 36 - protein, 16 aa, Dengue virus type 3
HRVDLFKPTSYGNGVY

SEQ ID NO: 37 - protein, 17 aa, Montana myotis leukoencephalitis virus
HRIDLFPPTSYGNGIIY

SEQ ID NO: 38 - protein, 14 aa, Rio Bravo virus
HRIDLFPPSSYGNG

SEQ ID NO: 39 - protein, 17 aa, Modoc virus
HRVDLFPPSSYGNGVLY

SEQ ID NO: 40 - protein, 16 aa, Sepik virus
HRVDLFPSTSYGNGIY

SEQ ID NO: 41 - protein, 16 aa, Alfuy virus
HRVDLYPVTSYGNGVY

SEQ ID NO: 42 - protein, 16 aa, Usutu virus
HRVDLYPETSYGNGVY

SEQ ID NO: 43 - protein, 16 aa, Murray Valley encephalitis virus
HRVDLYPITSYGNGVY

SEQ ID NO: 44 - protein, 16 aa, Japanese encephalitis virus
HRVDLYPRTSYGNGVY

SEQ ID NO: 45 - protein, 16 aa, Kedougou virus
HRVDLYPATSYGNGVY

SEQ ID NO: 46 - protein, 16 aa, Kunjin virus
HKVDLFPTTSYGNGVY

SEQ ID NO: 47 - protein, 16 aa, West Nile virus
HKVDLFPTTSYGNGVY

SEQ ID NO: 48 - protein, 16 aa, West Nile virus
HKVDLYPTTSYGNGVY

SEQ ID NO: 49 - protein, 16 aa, Zika virus
HKVDLYPATSYGNGVY

SEQ ID NO: 50 - protein, 16 aa, Yokose virus
HHVDLYPPTSYGNGIF

SEQ ID NO: 51 - protein, 16 aa, Bagaza virus
HNVDLYPTTSYGNGIF

SEQ ID NO: 52 - protein, 16 aa, Entebbe bat virus
HRVDLYPATSYGNGIF

SEQ ID NO: 53 - protein, 14 aa, Yellow fever vir
HRVDLYPSTSYGNG

SEQ ID NO: 54 - protein, 16 aa, Ilheus virus
HQVDLFPKTSYGNGIY

SEQ ID NO: 55 - protein, 16 aa, Rocio virus
HQVDLFPKTSYGNGLY

SEQ ID NO: 56 - protein, 16 aa, St. Louis encephalitis virus
HEVDLFPKTSYGNGVY

SEQ ID NO: 57 - protein, 17 aa, Langat virus
HRVDILAKTSYGNGLKY

SEQ ID NO: 58 - protein, 17 aa, Tick-borne encephalitis virus
HRVDILAKTSYGNGLKY

SEQ ID NO: 59 - protein, 17 aa, Louping ill virus
HRVDILAKTSYGNGPKY

SEQ ID NO: 60 - protein, 17 aa, Omsk hemorrhagic fever virus
HRVDILAKTSYGNGLRY

SEQ ID NO: 61 - protein, 17 aa, Powassan virus
HRVDILPRTSYGNGLKY

SEQ ID NO: 62 - protein, 17 aa, Karshi virus
HRIDFLPKTSYGNGLKY

SEQ ID NO: 63 - protein, 21 aa, Unknown Organism
  Description of Unknown Organism Viral peptide
VGLYGNGVVTRSGAYVSAIAN

SEQ ID NO: 64 - protein, 20 aa, Unknown Organism
  Description of Unknown Organism Viral peptide
VGLYGNGLKTNDTYVSSIAE

SEQ ID NO: 65 - protein, 20 aa, Unknown Organism
  Description of Unknown Organism Viral peptide
VGLYGNGIIHGDVYCSSIAQ

SEQ ID NO: 66 - protein, 20 aa, Unknown Organism
  Description of Unknown Organism Viral peptide
LGLYGNGLKSNDVYISSIAN

SEQ ID NO: 67 - protein, 20 aa, Unknown Organism
  Description of Unknown Organism Viral peptide
VGLYGNGPKTNESYVSSIAQ

SEQ ID NO: 68 - protein, 20 aa, Unknown Organism
  Description of Unknown Organism Viral peptide
VGLYGNGLKTNETYVSSIAQ

SEQ ID NO: 69 - protein, 20 aa, Unknown Organism
  Description of Unknown Organism Viral peptide
VGLYGNGLRTNETYVSSIAQ

SEQ ID NO: 70 - protein, 20 aa, Unknown Organism
  Description of Unknown Organism Viral peptide
VGLYGNGVLHGDTYCSSIAQ

SEQ ID NO: 71 - protein, 19 aa, Unknown Organism
  Description of Unknown Organism Viral peptide
VGLYGNGLKTKDTYVSGIA

SEQ ID NO: 72 - protein, 16 aa, Unknown Organism
  Description of Unknown Organism Viral peptide
TGLYGNGIVLYVSAIQ

SEQ ID NO: 73 - protein, 16 aa, Unknown Organism
  Description of Unknown Organism Viral peptide
IGLYGNGVILYVSAIQ

SEQ ID NO: 74 - protein, 16 aa, Unknown Organism
  Description of Unknown Organism Viral peptide
IGLYGNGVIMYISAIQ

SEQ ID NO: 75 - protein, 16 aa, Unknown Organism
  Description of Unknown Organism Viral peptide
IGLYGNGILTFVSAIQ

SEQ ID NO: 76 - protein, 16 aa, Unknown Organism
  Description of Unknown Organism Viral peptide
VGLYGNGVVTYVSAIN

SEQ ID NO: 77 - protein, 16 aa, Unknown Organism
  Description of Unknown Organism Viral peptide
IGLYGNGVVIYVSAIQ

SEQ ID NO: 78 - protein, 16 aa, Unknown Organism
  Description of Unknown Organism Viral peptide
IGLYGNGVIMYISAIQ

SEQ ID NO: 79 - protein, 16 aa, Unknown Organism
  Description of Unknown Organism Viral peptide
IGLYGNGVELYVSAIQ

SEQ ID NO: 80 - protein, 16 aa, Unknown Organism
  Description of Unknown Organism Viral peptide
VGLYGNGVVTYVSAIQ

SEQ ID NO: 81 - protein, 16 aa, Unknown Organism
  Description of Unknown Organism Viral peptide
VGLYGNGVLVYVSAIQ

SEQ ID NO: 82 - protein, 16 aa, Unknown Organism
  Description of Unknown Organism Viral peptide
IGLYGNGILTFVSSIQ

SEQ ID NO: 83 - protein, 16 aa, Unknown Organism
  Description of Unknown Organism Viral peptide
IGLYGNGVVTYVSAIQ

SEQ ID NO: 84 - protein, 16 aa, Unknown Organism
  Description of Unknown Organism Viral peptide
IGLYGNGILVFVSMIQ

SEQ ID NO: 85 - protein, 16 aa, Unknown Organism
  Description of Unknown Organism Viral peptide
IGLYGNGLVLYVSAIQ

SEQ ID NO: 86 - protein, 16 aa, Unknown Organism
  Description of Unknown Organism Viral peptide
VGLYGNGVVLYVSAIQ

SEQ ID NO: 87 - protein, 16 aa, Unknown Organism
  Description of Unknown Organism Viral peptide
IGLYGNGILIYVSAIQ

SEQ ID NO: 88 - protein, 16 aa, Unknown Organism
  Description of Unknown Organism Viral peptide
VGLYGNGVILYVSAIQ

SEQ ID NO: 89 - protein, 16 aa, Unknown Organism
  Description of Unknown Organism Viral peptide
IGLYGNGVIMYISAIQ

SEQ ID NO: 90 - protein, 16 aa, Unknown Organism
  Description of Unknown Organism Viral peptide
VGLYGNGVVTYVSGIQ

SEQ ID NO: 91 - protein, 16 aa, Unknown Organism
  Description of Unknown Organism Viral peptide
IGLYGNGVLIYVSGIQ

SEQ ID NO: 92 - protein, 13 aa, Unknown Organism
  Description of Unknown Organism Viral peptide
IGLYGNGVSAIST

SEQ ID NO: 93 - protein, 13 aa, Unknown Organism
  Description of Unknown Organism Viral peptide
IGLYGNGCSSIAT

SEQ ID NO: 94 - protein, 17 aa, Dengue virus type 2
HRVDLFSPTSYGNGVVY

SEQ ID NO: 95 - protein, 16 aa, West Nile virus
HKVDLYPTTSYGNGVY

SEQ ID NO: 96 - protein, 16 aa, Kunjin virus
HKVDLFPTTSYGNGVY

SEQ ID NO: 97 - protein, 16 aa, Zika virus
HKVDLYPATSYGNGVY

SEQ ID NO: 98 - protein, 16 aa, Alfuy virus
HRVDLYPVTSYGNGVY

SEQ ID NO: 99 - protein, 16 aa, Murray Valley encephalitis virus
HRVDLYPITSYGNGVY

SEQ ID NO: 100 - protein, 16 aa, Unknown Organism
  Description of Unknown Organism Viral peptide
HRVDLFSPTSYGNGVY

SEQ ID NO: 101 - protein, 16 aa, Usutu virus
HRVDLYPETSYGNGVY

SEQ ID NO: 102 - protein, 16 aa, Kedougou virus
HRVDLYPATSYGNGVY

SEQ ID NO: 103 - protein, 16 aa, Ilheus virus
HQVDLFPKTSYGNGIY

SEQ ID NO: 104 - protein, 16 aa, St. Louis encephalitis virus
HEVDLFPKTSYGNGVY

SEQ ID NO: 105 - protein, 16 aa, Dengue virus type 4
HRVDLFKPTSYGNGVY

SEQ ID NO: 106 - protein, 16 aa, Rocio virus
HQVDLFPKTSYGNGLY

SEQ ID NO: 107 - protein, 16 aa, Dengue virus type 1
HRVDLFKPTSYGNGVY

SEQ ID NO: 108 - protein, 16 aa, Dengue virus type 3
HRVDLFKPTSYGNGVY

SEQ ID NO: 109 - protein, 16 aa, Yokose virus
HHVDLYPPTSYGNGIF

SEQ ID NO: 110 - protein, 14 aa, Yellow fever virus
HRVDLYPSTSYGNG

SEQ ID NO: 111 - protein, 17 aa, Langat virus
HRVDILAKTSYGNGLKY

SEQ ID NO: 112 - protein, 17 aa, Omsk hemorrhagic fever virus
HRVDILAKTSYGNGLRY

SEQ ID NO: 113 - protein, 17 aa, Tick-borne encephalitis virus
HRVDILAKTSYGNGLKY

SEQ ID NO: 114 - protein, 17 aa, Louping ill virus
HRVDILAKTSYGNGPKY

SEQ ID NO: 115 - protein, 16 aa, Entebbe bat virus
HRVDLYPATSYGNGIF

SEQ ID NO: 116 - protein, 16 aa, Sepik virus
HRVDLFPSTSYGNGIY

SEQ ID NO: 117 - protein, 17 aa, Karshi virus
HRIDFLPKTSYGNGLKY

SEQ ID NO: 118 - protein, 16 aa, Japanese encephalitis virus
HRVDLYPRTSYGNGVY

SEQ ID NO: 119 - protein, 14 aa, Rio Bravo virus
HRIDLFPPSSYGNG

SEQ ID NO: 120 - protein, 17 aa, Montana myotis leukoencephalitis virus
HRIDLFPPTSYGNGIIY

SEQ ID NO: 121 - protein, 17 aa, Modoc virus
HRVDLFPPSSYGNGVLY

SEQ ID NO: 122 - protein, 16 aa, Bagaza virus
HNVDLYPTTSYGNGIF

SEQ ID NO: 123 - protein, 17 aa, Powassan virus
HRVDILPRTSYGNGLKY

(19) United States
(12) Patent Application Publication
(10) Pub. No.: US 2008/0059077 A1
(43) Pub. Date: Mar. 6, 2008

(54) Methods and systems of common motif and countermeasure discovery

(75) Inventors: Carol E. Zhou, Pleasanton, CA (US); Adam T. Zemla, Brentwood, CA (US); Diana C. Roe, Newark, CA (US); Joseph P. Schoeniger, Oakland, CA (US)

Correspondence Address: Lawrence Livermore National Security, LLC, LAWRENCE LIVERMORE NATIONAL LABORATORY, PO BOX 808, L-703, LIVERMORE, CA, 94551-0808, US

(73) Assignee: The Regents of the University of California, Oakland, CA (US)

(21) Appl. No.: 11/818,075

(22) Filed: Jun. 12, 2007

Related U.S. Application Data
(60) Provisional application No. 60/813,211, filed on Jun. 12, 2006.

(51) Int. Cl. G01N 33/48; G06G 7/48

(52) U.S. Cl. 702/19; 703/11

(57) ABSTRACT

Disclosed are computational methods, and associated hardware and software products for identifying a set of target pockets for broad-spectrum drug development based on a provided set of protein motifs. A method of identifying the provided set protein motifs based on a plurality of protein motifs in also disclosed herein. Additional methods for generating a plurality of protein motifs based on both aligned protein structure and sequences are disclosed herein.

### CROSS REFERENCE TO RELATED APPLICATION

This application claims the benefit of Provisional Application No. 60/813,211 filed Jun. 12, 2006, the entire disclosure of which is hereby incorporated by reference, in its entirety.

### STATEMENT REGARDING FEDERALLY FUNDED RESEARCH

The United States Government has rights in this invention pursuant to Contract No. W-7405-ENG-48 between the United States Department of Energy and the University of California, for the operation of Lawrence Livermore National Laboratory.

### TECHNICAL FIELD

The disclosed embodiments generally relate to the bioinformatics and chemoinformatics discovery of motifs and ligand molecules that bind them. Specifically, the disclosed embodiments relate to methods of discovering and characterizing protein structural motifs that are targets for development of reagents with wide-range, robust, or highly specific activity.

### BACKGROUND

Evolution is a naturally occurring phenomenon in all organisms including humans and human pathogens such as viruses, bacteria and other microorganisms, and by which mutations are created in the sequences of deoxyribose nucleic acid (DNA) or ribonucleic acid (RNA) that encodes the organism's genetic information. The pattern of mutations in genes encoding polypeptide products determines the interconnected structural and chemical features of the gene product that, in turn, determine its function. These patterns may differ or be conserved between individual organisms or across species at the nucleic acid sequence level in a manner that does or does not produce significant differences in the features of the corresponding gene product. For example, specific changes in viral sequences may have either minimal effects on function or may confer resistance to a drug by altering the binding sites of the protein of the organism that the drug binds to. Drug resistance may also be created by genetic manipulation of viruses and pathogens for use as biological weapons. On the other hand, a small number of differences in sequence between two genes in an organism may determine highly significant structural and functional differences that are conserved across many species (e.g., cyclooxygenase type I versus type II).

The development of broad-spectrum therapeutics that have a wide range of activity over different individual or groups of organisms, regardless of differences between them generated by evolution or genetic manipulation, has become a favored counter measure strategy against naturally occurring, emerging or man-made biological threats. On the other hand, the discovery of drug molecules that target only certain conserved features of a protein enables development of therapeutics with highly specific activity across a range of individuals and species. Identifying, within potential protein drug targets, potential binding sites that are conserved across a diverse set of target organisms therefore enables development of broad-spectrum therapeutics as well as development of highly selective therapeutics (drugs may be both).

Compound screening provides one non-computational or “wet lab” method of identifying a set of binding sites in a set of proteins for targeting broad-spectrum therapeutic development. In addition to being costly and time-consuming, wet lab methods are based on the principle of discovery and provide no a priori quantitative characterization of the protein residues which may form a binding site for the compounds. Consequently, traditional methods based on compound library screens provide little information that can be used for down-selecting or targeting the possible pool of reagents.

Computational or bioinformatics methods of motif generation often use conservation as criteria in evaluating functionally important residues in both protein sequences and protein structures. Conservation is the phenomena in which residues in a protein are not subject to mutation due to functional importance. Homologous sequences are defined as sequences that occur through duplication, either through evolution or within an organism.

Alignments between homologous sequences are used to identify motifs that are thought to have functional significance through conservation. A more stringent method of identifying motifs for therapeutic development is through the identification of structural conservation (Zhou et. al, Bioinformatics) in homologous structures. A fundamental limitation of both sequence and structure based methods of determining motifs is that they only generate single motifs, which are not characterized relative to each other or to the proteomes of the target organisms. A proteome is the complete set of proteins that an organism expresses. Conservation can vary between different proteins, causing motifs generated from some proteins to have greater applicability from broad-spectrum therapeutic development than others. Therefore, the evaluation of a motif as a candidate for a broad spectrum therapeutic cannot be performed independently of the evaluation of other motifs.

Chemoinformatics approaches apply computational techniques to problems in the field of chemistry. Chemoinformatics provide alternate and complementary approaches to bioinformatics based identification of broad-spectrum targets through the characterization of molecular interactions. Chemoinformatics approaches to identifying therapeutics based on protein structure include computational methods such as Structure Activity Relationship analysis for characterizing the binding activity of proteins for therapeutic development. Chemoinformatics programs such as UniquePocket (Zhou, et al. Bioinformatics) provide the identification of concavities on the surface of a protein structure which may form active binding sites. Chemoinformatics programs are also available to provide computational simulations of docking small molecules to the active binding sites which enable the characterization of a pocket or active binding site by the molecules the active binding site binds to. While chemoinformatics methods greatly facilitate drug discovery, they also suffer from the same limitations as bioinformatics approaches when applied to discovery of broad-range therapeutics. That is, the evaluation typically occurs on the basis of an individual motif instead of a complete set of motifs which should be studied relation to each other when derived from a set of target organisms.

The same shortcomings in motif evaluation apply in the characterization of candidates for broad-spectrum therapeutics. Without information regarding binding activity of a therapeutic relative both to target proteins and to other therapeutics, it is impossible to characterize the therapeutic for broad-spectrum therapeutic development.

### SUMMARY OF THE INVENTION

These needs are met by methods and computer program products for identifying a set of target pockets for broad-spectrum drug development, wherein each target pocket comprises a three-dimensional concavity in a protein structure.

In one aspect, the present invention provides a computer-implemented method for identifying a set of target pockets for broad-spectrum drug development, wherein each target pocket comprises a three-dimensional concavity in a protein structure. Initially, the method is provided with a set of protein motifs, wherein each protein motif of the set of motifs comprises a first plurality of conserved residues. The method identifies a plurality of pockets based on the set of protein motifs, wherein each pocket comprises a second plurality of conserved residues that define the three dimensional concavity on the surface of a protein structure, wherein the second plurality of conserved residues correspond at least in part with the first plurality of conserved residues. The method further generates a plurality of binding profiles in association with the plurality of pockets, wherein each binding profile specifies at least one calculated binding activity between each pocket and at least one test molecule. The method further generates a plurality of pocket similarity values based on the plurality of binding profiles, wherein each pocket similarity value is based on binding profiles associated with at least two pockets of the plurality of pockets. The method further identifies and stores a set of target pockets based on the plurality of pocket similarity values.

In another aspect, the present invention provides a computer-implemented method of providing a set of protein motifs.

In another aspect, the present invention provides computer-implemented methods of identifying a plurality of motifs.

In another aspect the present invention provides a computer-readable storage medium encoded with program code for identifying a set of target pockets for broad-spectrum drug development, wherein each target pocket comprises a three-dimensional concavity in a protein structure. Initially, the method is provided with a set of protein motifs, wherein each protein motif of the set of motifs comprises a first plurality of conserved residues. The method identifies a plurality of pockets based on the set of protein motifs, wherein each pocket comprises a second plurality of conserved residues that define the three dimensional concavity on the surface of a protein structure, wherein the second plurality of conserved residues correspond at least in part with the first plurality of conserved residues. The method further generates a plurality of binding profiles in association with the plurality of pockets, wherein each binding profile specifies at least one calculated binding activity between each pocket and at least one test molecule. The method further generates a plurality of pocket similarity values based on the plurality of binding profiles, wherein each pocket similarity value is based on binding profiles associated with at least two pockets of the plurality of pockets. The method further identifies and stores a set of target pockets based on the plurality of pocket similarity values.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a system architecture adapted to support one embodiment.

FIG. 2 is a block diagram illustrating a Protein Set Selection Engine according to one embodiment.

FIG. 3. is a block diagram illustrating a Sequence Motif Analysis Engine according to one embodiment.

FIG. 4. is a block diagram illustrating Structure Motif Analysis Engine according to one embodiment.

FIG. 5. is a block diagram illustrating a Protein Pocket Engine according to one embodiment.

FIG. 6. is a flow chart illustrating the high level steps of target motif and molecule identification according to one embodiment.

FIG. 7. is a flow chart illustrating the high level steps of sequence and structure motif generation according to one embodiment.

FIG. 8 is a flow chart illustrating the high level steps of sequence motif quantitative structure activity relationship determination according to one embodiment.

FIG. 9 is a diagram illustrating an overview of a working example of the system.

FIG. 10(a) is a graphic illustration of a pair-wise structural alignment of 7 homologous proteins with serine protease from Dengue virus (PDB code: 2fom_B) using the Local Global Alignment (LGA) program, as described in US Patent Application Number 2004/0185486.

FIG. 10(b) illustrates a 3D representation of the superimposed structures of Dengue virus serine protease (PDB code: 2fom_B) and West Nile Virus serine protease (PDB code: 2ijo_B).

FIG. 10(c) illustrates a 3D representation of two superimposed structures of Dengue virus serine protease (2fom_B and 1df9).

FIG. 11 is a graphic illustration of a portion of a multiple structure alignment between the protein structures of serine protease of the Dengue virus and 30 homologous protein structures.

FIG. 12(a) is a graphic illustration of a structure alignment of a structural motif identified based on the alignment illustrated in FIG. 11 and identification of active binding sites using Common Pocket.

FIG. 12(b) is a graphic illustration of a sequence alignment generated based on the sequences associated with protein sequences associated with the structural protein motif in FIG. 12(a).

FIG. 13 is a dendogram created by clustering the Flavivirus sequences based on sequence and structural motif alignments illustrated in FIGS. 12(a) and 12(b).

FIG. 14 is a “ribbon diagram” of four representative flavivirus protease structures, each taken from a separate cluster based on the structure/sequence/motif alignment.

FIG. 15 illustrates the identification of pockets in the protein structures of the Dengue, Langat, West Nile and Yellow Fever viruses.

FIG. 16 is a graphic illustration of the clustering of protein motifs in homologous proteins based on binding profiles.

The figures depict various embodiments of the present invention for purposes of illustration only. One skilled in the art will readily recognize from the following discussion that alternative embodiments of the structures and methods illustrated herein may be employed without departing from the principles of the invention described herein.

### DEFINITIONS

Residue: An amino acid residue is one amino acid that is joined to another by a peptide bond. Residue is referred to herein to describe both an amino acid and its position in a polypeptide sequence.

Motif: A motif is a set of residues in a protein that are conserved in the structure or sequence of a set of homologous proteins. Motifs may also form three dimensional structures which confer functional properties in the set of homologous proteins.

Surface residue: A surface residue is a residue located on a surface of a polypeptide. In contrast, a buried residue is a residue that is not located on the surface of a polypeptide. A surface residue usually includes a hydrophilic side chain. Operationally, a surface residue can be identified computationally from a structural model of a polypeptide as a residue that contacts a sphere of hydration rolled over the surface of the molecular structure. A surface residue also can be identified experimentally through the use of deuterium exchange studies, or accessibility to various labeling reagents such as, e.g., hydrophilic alkylating agents.

Polypeptide: A single linear chain of 2 or more amino acids. A protein is an example of a polypeptide.

Homolog: A gene related to a second gene by descent from a common ancestral DNA sequence. The term, homolog, may apply to the relationship between genes separated by the event of speciation or to the relationship between genes separated by the event of genetic duplication. Organisms that un-related or distantly related though evolution may contain homologous sequences due to convergent evolution or targeted manipulated of their genetic material.

Taxonomy: The classification of organisms in an ordered system that indicates natural relationships. As discussed herein, taxonomy is a classification of organisms that indicates evolutionary relationships.

Conservation: Conservation is a high degree of similarity in the primary or secondary structure of molecules between homologs. This similarity is thought to confer functional importance to a conserved region of the molecule. In reference to an individual residue or amino acid, conservation is used to refer to a computed likelihood of substitution or deletion based on comparison with homologous molecules.

Distance Matrix: The method used to present the results of the calculation of an optimal pair-wise alignment score. The matrix field (i,j) is the score assigned to the optimal alignment between two residues (up to a total of i by j residues) from the input sequences. Each entry is calculated from the top-left neighboring entries by way of a recursive equation.

Substitution Matrix: A matrix that defines scores for amino acid substitutions, reflecting the similarity of physicochemical properties, and observed substitution frequencies. These matrices are the foundation of statistical techniques for finding alignments.

### DETAILED DESCRIPTION OF THE DRAWINGS

Embodiments are now described with reference to the figures where like reference numbers indicate identical or functionally similar elements.

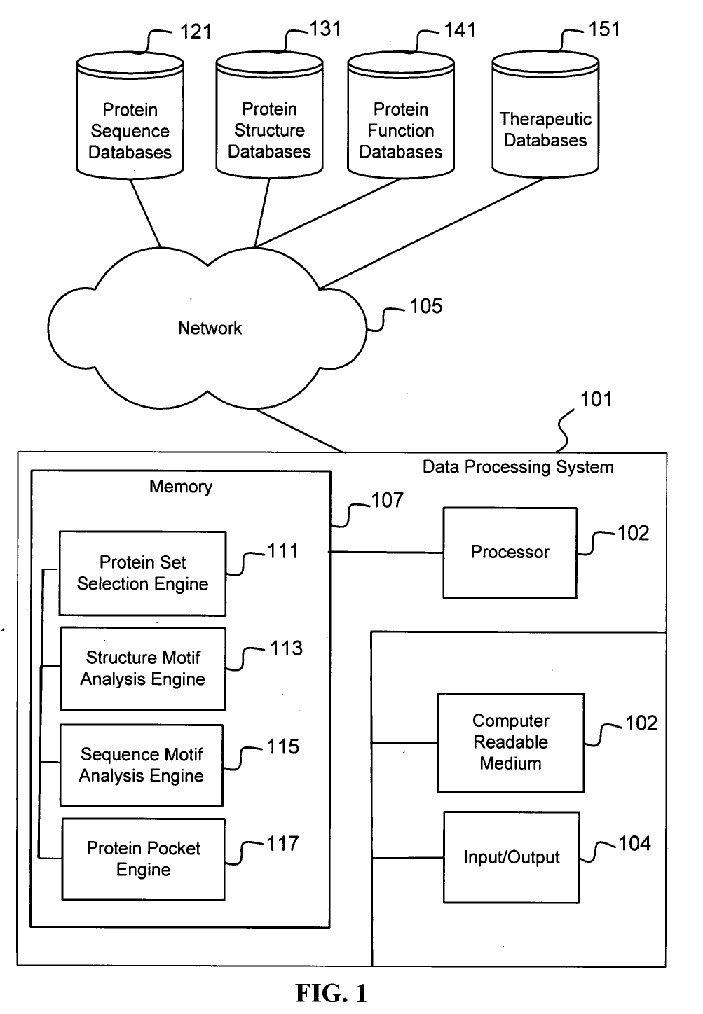

FIG. 1 shows a system architecture 100 adapted to support one embodiment. FIG. 1 shows components used to generate and characterize protein motifs. The system architecture 100 includes a network 105, through which any number of Protein Structure Databases 131, Function Databases 141, Sequence Databases 121 and Therapeutic Databases 151 are accessed by a data processing system 106.

FIG. 1 shows component engines used to generate and characterize protein motifs. The data processing system 101 includes a Protein Set Selection Engine 111, a Structure Motif Analysis Engine 113, a Sequence Motif Analysis Engine 115 and a Protein Pocket Engine 117. Each of the foregoing is implemented, in one embodiment, as software modules (or programs) executed by processor 116.

The Protein Set Selection Engine 111 operates to identify a set of proteins for future analysis 101 by accessing the Protein Sequence Databases 121 and Protein Structure Databases 131 through the network 105. (as operationally and programmatically defined within the data processing system)

The Sequence Motif Analysis Engine 115 operates to identify sequence motifs from a set of protein sequences. In one embodiment, the Sequence Motif Analysis Engine 115 operates in communication with the Protein Set Selection Engine 111. In another embodiment, the Sequence Motif Analysis Engine 115 accesses the Protein Sequence Databases 121 through the Network 105 to select a group of sequences. The Sequence Motif Analysis Engine 115 also operates to identify a set of therapeutics with common binding activity to sequence motifs. According to the embodiment, the Sequence Motif Analysis Engine 115 accesses the Therapeutic Databases 151 directly to import a set of therapeutics.

The Structure Motif Analysis Engine 113 operates to identify structure motifs from a set of protein structures. In one embodiment, the Structure Motif Analysis Engine 113 accesses protein structures from the Protein Structure Database 131. According to the embodiment, the Structure Motif Analysis Engine 113 accesses the Protein Sequence Database 121 to obtain protein sequences.

The Protein Pocket Engine 117 operates to determine whether motifs identified by the Structure Motif Analysis Engine 113 and the Sequence Motif Analysis Engine 115 form a pockets or three dimensional concavities on the surface of the protein structure. The Protein Pocket Engine 117 characterizes the spatial relationships between motif residues in a protein structure to determine whether they form a pocket or concavity that facilitates drug binding. The Protein Pocket Engine 177 also operates to generate a binding profiles based on computational docking of small molecules to the pockets. The Protein Pocket Engine 177 further operates to cluster a set of motifs and a set of molecules based on the generated binding profiles.

According to the embodiment of the present invention, the Protein Set Selection Engine 111, the Structure Motif Analysis Engine 113, the Sequence Motif Analysis Engine 115 and the Protein Pocket Engine 117 can operate on the sequences, structures and/or motifs in different orders than described above.

It should also be appreciated that in practice at least some of the components of the data processing system 101 will be distributed over multiple computers, communicating over a network. For example, the Sequence Motif Analysis Engine 115 may be deployed over multiple servers. As another example, the Protein Pocket Engine 117 may be located on any number of different computers. For convenience of explanation, however, the components of the data processing system 101 are discussed as though they were implemented on a single computer.

In another embodiment, some or all of the Protein Sequence Databases 121, the Protein Structure Databases 131, the Protein Function Databases 141 and the Therapeutics Databases 151 are located on the data processing system 101 instead of being coupled to the data processing system 101 by a network 105. For example, the Protein Set Selection Engine 111 may import protein sequence from Protein Sequence Databases 131 that are a part of or associated with the data processing system 101.

FIG. 1 shows that the data processing system 101 includes a memory 107 and one or more processors 102. The memory 107 includes the Protein Set Selection Engine 111, the Structure Motif Analysis Engine 113, the Sequence Motif Analysis Engine 115, and the Protein Pocket Engine 117 each of which is preferably implemented as instructions stored in memory 107 and executable by processor 102.

FIG. 1 also includes a computer readable medium 118 containing, for example, at least one of the Protein Set Selection Engine 111, the Structure Motif Analysis Engine 113, the Sequence Motif Analysis Engine 115, and the Protein Pocket Engine 117. FIG. 1 also includes one or more input/output devices 104 that allow data to be input and output to and from the data processing system 101. It will be understood that embodiments of the data processing system 101 also include standard software components such as operating systems and the like and further include standard hardware components not shown in the figure for clarity of example.

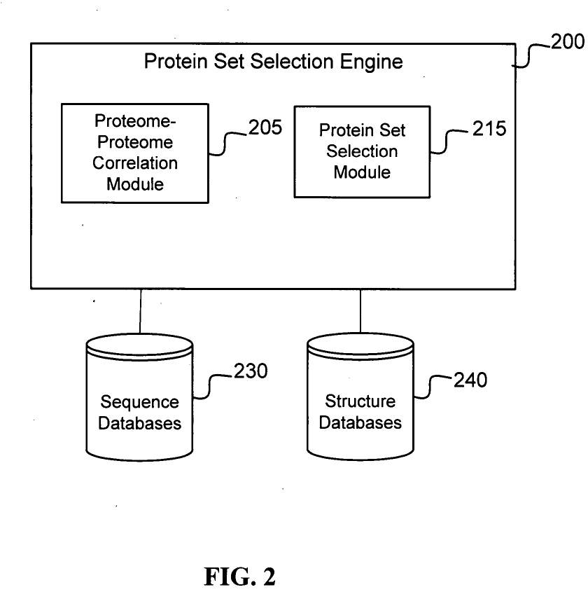

FIG. 2. is a block diagram illustrating the Protein Set Selection Engine 200 according to one embodiment. The Protein Set Selection Engine 200 is used to select a set of proteins for common motif detection. The Protein Set Selection is adapted to communicate with Protein Sequence Databases 230 such as MvirDB or MannDB (Zhou et al. NAR, Zhou et al. BMC Bioinformatics) or GenBank (available at the website of the National Center for Biotechnology Information). According to the embodiment, the Protein Set Selection Engine 200 may be adapted to import sequences from Protein Structure Databases 240 such as Protein DataBank (PDB, available at the website of the Research Collaboratory for Structural Bioinformatics). In one embodiment, the Protein Set Selection Engine may directly access the Protein Structure Databases 240 and the Protein Sequence Databases 230. In other embodiments, the Protein Set Selection Engine 200 may access the Protein Structure Databases 240 and the Protein Sequence Databases 230 through a network.

The Protein Set Selection Engine 200 consists of two modules, a Proteome-Proteome Correlation Module 205 and a Protein Set Selection Module 215. The functions of the engines discussed herein are separated into modules for purposes of discussion only. Different embodiments of the present invention may distribute functions among modules in different ways.

A proteome is defined as the set of proteins produced (or predicted to be produced) by an organism. The Proteome-Proteome Correlation Module 205 is used to determine the set of homologous proteins between proteins or proteomes of different organisms targeted for therapeutic development.

Target organisms or proteins the Proteome-Proteome Correlation Module 205 uses to determine the set of homologous proteins may be specified in a number of different ways based on user input. In one embodiment, a user can manually pre-select a group of target organisms by any pre-defined criterion such as taxonomy or phylogeny. In some embodiments, the user may specify a target organism or protein and a cutoff specifying a minimum or maximum value of a sequence or structure alignment to determine a set of homologous proteins for a given proteome. In another embodiment, the Proteome-Proteome Correlation Module 205 is adapted to receive a set of proteins from the Protein Set Selection Module 215. Other methods of selecting a set of target proteins are discussed in detail below in the section titled Near-Neighbor and Target Selection.

According to the embodiment, the Proteome-Proteome Correlation Module 205 may evaluate proteins or proteomes as protein sequences imported from Protein Sequence Databases 230 such as GenBank or InterPro (available at the website of the European Bioinformatics Institute). In this embodiment, the Proteome-Proteome Correlation Module 205 performs pair-wise sequence alignments between the sets of sequences defining the proteomes of target organisms using programs such as BLAST, FASTA, or HMMer (Atschul et al., 1990, McClure et al., 1996, and Pearson et al., 1998) to determine the set of homologous protein sequences between the proteins or proteomes.

In some embodiments, the Proteome-Proteome Correlation Module 205 generates protein structures using protein sequences from Protein Sequence Databases 230 such as GenBank. According to the embodiment, protein structures may be generated in many ways. In one embodiment, the Proteome-Proteome Correlation Module 205 generates protein structures using the AS2TS system (Zemla et al., 2005). Various different methods of protein structure modeling are discussed in detail below in the section titled Protein Structure Modeling. Accordingly, the Proteome-Proteome Correlation Module 205 generates structural alignment to determine structural homologs between proteins with solved protein structure. Various methods of structural alignment are discussed in detail below in the section titled Protein Structure Alignment.

The Protein Set Selection Module 215 is used to pre-select sets of protein sequences for further processing based on input criteria such as target organisms, virulence factors, toxins and functional properties of the protein. According to the embodiment, the Protein Set Selection Module 215 may use function annotations of protein sequence in selecting sets of proteins such as those used in MannDB (available at the website of Lawrence Livermore National Laboratory). These functional annotations are discussed in detail with respect to MannDB in Zhou et al, BMC Bioinformatics 2006, hereby incorporated by reference. According to the embodiment, the Protein Set Selection Module 215 may take as input one or more target protein structures or sequences or target organisms. Methods of determining a set of target and near neighbor organisms or sequences are discussed in detail below in the section titled Near-Neighbor and Target Selection.

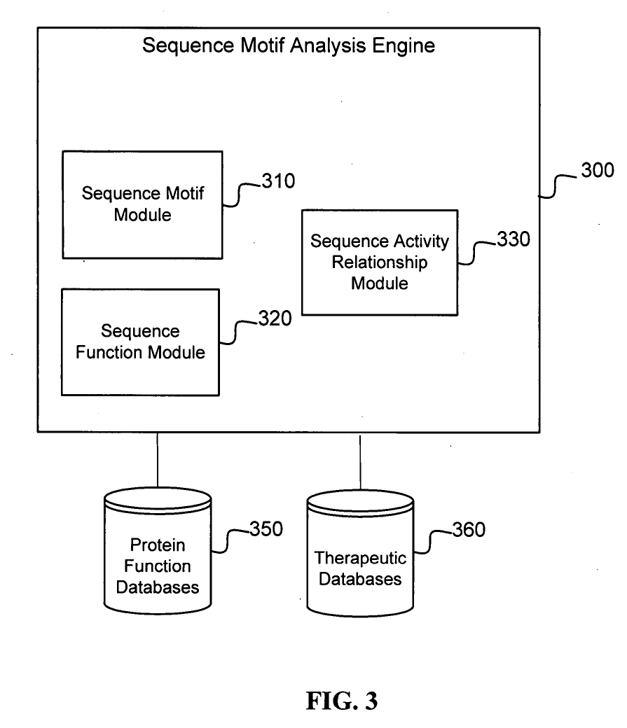

FIG. 3. is a block diagram illustrating the Sequence Motif Analysis Engine 300 according to one embodiment. The Sequence Motif Analysis Engine 300 determines sequence motifs in homologous proteins in a set of target organisms. In one embodiment, the Sequence Motif Analysis Engine is adapted to receive a set of homologous protein sequences from the Protein Set Selection Engine 200. The Sequence Motif Analysis Engine 300 may also receive sets of homologous proteins from other sources.

The Sequence Motif Analysis Engine 300 contains a Sequence Motif Module 310, a Sequence Function Module 320 and a Sequence Activity Relationship Module 330. The Sequence Motif Module 310 identifies common and conserved motifs in homologous protein sequences. In one embodiment, the Sequence Motif Module 310 uses a multiple sequence alignment algorithm such as CLUSTALW (Higgins et al., 1994) or multi-FASTA (Schwartz and Pachter, 2007) to generate a multiple sequence alignment of a set of homologous subsequences from which to determine motifs. In another embodiment, the Sequence Motif Module 310 clusters a set of protein sequences in order to generate a set of homologous subsequences.

In one embodiment, the Sequence Motif Module 310 clusters the sequences based on local sequence alignment, a global sequence alignment or a local-global sequence alignment. Any kind of sequence clustering algorithm can be employed by the Sequence Motif Module 310 to cluster the sequences such as single linkage clustering approaches (Petryszak at al., 2005, Gront and Kolinski, 2005). Other sequence clustering algorithms will be apparent to those skilled in the art.

According to the embodiment of the present invention, the Sequence Motif Module 310 generates a set of scores based on a similarity metric which measures conservation at different residues. In some embodiments, similarity metrics are based on systems that define residue substitution rates, such as protein alphabets or pre-defined matrices such as BLOSUM (Heinkoff and Heinkoff, 1992). These as well as other applicable similarity metrics are discussed in detail below in the section titled Similarity Metrics. Methods of scoring residues for conservation are discussed in the section titled Conservation Uniqueness Score.

In other embodiments, the Sequence Motif Module 310 identifies motifs using Hidden Markoff Model algorithms such as HMMer (available at the website of the Pittsburgh Supercomputing Center). Other methods of characterizing conservation within a set of aligned protein sequences in order to identify functional motifs such as Gibbs sampling (Chen and Jiang, 2006) and Bayesian methods (Jensen and Liu, 2004) or graph theoretic approaches of identifying motifs (Naughton et al., 2006) can also be applied in the present invention. It is expected that other algorithms for determining sequence motifs from a set of homologous sequences will be apparent to those skilled in the art.

The Sequence Function Module 320 determines annotations of functional properties associated with one or more residues in a motif using information derived from Protein Function Databases 350. In most embodiments, the motifs annotated by the Sequence Function Module 320 will be generated by the Sequence Motif Module 310. In some embodiments, the motifs annotated by the Sequence Function Module 320 will be generated by the Structure Motif Module 410. The Sequence Function Module 320 is also adapted to receive motifs from known databases of protein motifs such as InterPro (available at the website of the European Bioinformatics Institute). The Sequence Function Module 320 is adapted to communicate with Protein Function Databases 350 such as MvirDB and MannDB (both available at the Lawrence Livermore National Laboratory website) directly or through a network. The Sequence Function Module 320 accesses protein sequence functional annotations from the Protein Function Databases 350 at both the sequence and the domain levels.

Any system of functional annotation of sequence or structural elements can be used by the Sequence Function Module 320 to associate sequence motifs with associated functional information. In one embodiment, the Sequence Function Module 320 associates sequence motifs with annotations of sequences associated with known drug target databases such as DrugBank (Wishart et al., 2006). In another embodiment, the Sequence Function Module 320 associates sequence motifs with information regarding known enzyme targets available from the BRENDA database (Schomburg et al., 2004). According to the embodiment, the Sequence Function Module may use function annotations of protein sequence such as those in MannDB. It is expected that other systems of annotation of protein sequences will be apparent to those skilled in the art.

The Sequence Activity Module 330 evaluates the binding activity of sequence motifs to identify potential lead drug compounds. In one embodiment, the Sequence Activity Module 330 receives a set of motifs annotated with known drug enzymes or targets from the Sequence Function Module 320.

In some embodiments, the Sequence Activity Module 320 is adapted to communicate with the MDL Drug Data Report-3D database (MDDR-3D, MDL and Prious Bioscience, 2007). The MDDR-3D database contains over 165,000 known drug targets and compounds. The Sequence Activity Module cross-references molecules from MDDR-3D with databases such as BRENDA and DrugBank based on analysis of chemical similarity between the molecules in the databases.

Analysis of chemical similarity between drug molecules can be performed using a number of different algorithms. In one embodiment, the signature descriptor (Faulon et al., 2004) is used to represent molecule data as feature vectors in order to facilitate similarity analyses. The signature descriptor is discussed in detail below. Other applicable methods of analyzing inter-molecular structural similarity between drug molecules include reaction indexing, pharmacophore matching, database clustering, diversity analysis and virtual screening (Willett. 2005, McMartin and Bohacek, 2004, Waszkowycz et al. 2001, Bayley et al., 1999).

In some embodiments, the Sequence Activity Module 330 uses the list of cross-referenced drug targets to expand the list of known targets from DrugBank and BRENDA to associate the set of motifs from the Sequence Function Module 320. The Sequence Activity Module 330 searches the table of cross-referenced molecules and adds molecules from MDDR-3D to the list of known targets for a motif based on similarity to molecules in DrugBank and BRENDA.

The Sequence Activity Module 330 generates Quantitative Structure Activity Relationship (QSAR) analyses to predict binding of identified motifs to the set of molecules identified as drug targets that are associated with the motifs. In one embodiment, the QSAR analysis is based on a signature descriptor (Faulon, 2004). Signature descriptors and other applicable methods of QSAR analysis are discussed in detail below in the section title Quantitative Structure Activity Relationship Modeling.

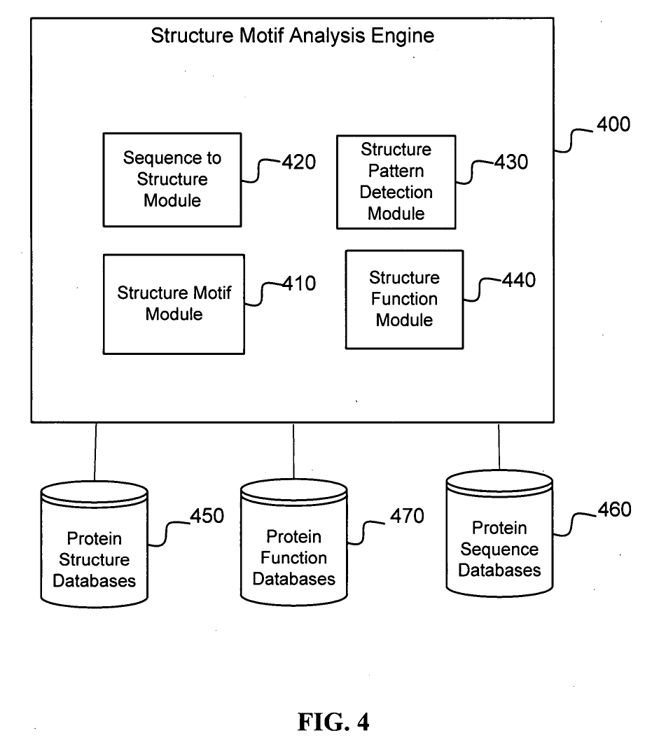

FIG. 4. is a block diagram illustrating the Protein Structure Motif Engine 400 according to one embodiment. The Protein Structure Motif Engine 400 consists of four modules, a Sequence to Structure Module 420, a Structure Motif Module 410, a Structure Pattern Detection Module 430 and a Structure Function Module 440. In some embodiments, the Structure Motif Analysis 400 may be adapted to communicate with the Sequence Motif Analysis Engine 300.

The Sequence to Structure Module 420 computationally models three dimensional protein structures from protein sequence. In one embodiment, the Sequence to Structure Module 420 accesses the Protein Sequence Databases 460 and Protein Structure Databases 450 directly to import protein sequences and protein structures. In other embodiments the Sequence to Structure Module receives sets of target or near-neighbor protein sequence or structure from the Protein Set Selection Engine 200.

According to the embodiment, the Sequence to Structure Module 420 can use any method of protein structure modeling such as ab-initio modeling, threading or sequence-sequence based methods of fold recognition. These and other methods of protein structure modeling are discussed in detail below in the section title Protein Structure Modeling. In one embodiment, the Sequence to Structure Module 420 uses the AS2TS system of protein structure modeling.

In some embodiments, the Sequence to Structure Module 420 may use a sequence alignment in combination with a threshold protein sequence similarity in order to determine a set of protein sequences for which to model protein structure. In this embodiment, the Sequence to Structure Module 420 generates sequence alignments for the set of sequences to be modeled with sequences of proteins with solved empirical structure (crystal or nuclear magnetic resonance) in the Protein Structure DataBank. If the sequences to be modeled have a sufficient similarity to one or more sequences with known protein structure, then the Sequence to Structure Module 420 will model the three dimensional structure of the sequence.

The Structure Pattern Detection Module 430 generates one-to-one residue correspondences for aligned sets of protein structures. These correspondences are herein referred to as “spans”.

According to the embodiment, the Structure Pattern Detection Module 430 may generate spans using multiple protein structure alignments of a set of protein structures or by combining multiple pair-wise alignments of protein structures. Suitable algorithms for generating protein structure alignments are discussed below in the section titled Protein Structure Alignment. In one embodiment, a Local-Global Alignment (Zemla, NAR 2003) is used to generate a multiple protein structure alignment. In some embodiments, the Structure Motif Module 410 creates a multiple protein structure alignment of both the target and the near-neighbor sets of protein structures.

In one embodiment, the Structure Pattern Detection Module 430 generates spans in homologous proteins by clustering protein structures using Structure Alignment-based Clustering of Proteins (STRALCP, Zemla et al. in preparation, see also Provisional Application No. 60/813,211). STRALCP uses Local-Global Alignment structural alignment techniques to identify local and global similarities within sets of proteins. Local Global Alignment techniques are discussed below. Conserved sub-structures or ‘spans’ are determine based on local similarities and are used along with global similarity of protein structures to cluster the proteins. According to the embodiment, spans common to a cluster of proteins may be grouped by the Structure Pattern Detection Module 430 as homologous sequences and communicated to the Structure Motif Module 410 for further analyses.

In other embodiments spans or domains may be identified by the Structure Pattern Detection Module 430 using a pre-computed classification of protein structures such as Structural Classification of Proteins (SCOP, available at the website of the University of California, Berkeley) or the Class Architecture Topology Homology database (Pearl et al., 2005). SCOP protein structures are manually classified as to structural and evolutionary relatedness. In CATH, structures are grouped or clustered according to secondary structure (class), gross orientation of secondary structure (architecture), topology (folds and connections) and homology. Using databases of pre-clustered proteins, the Structure Pattern Detection Module 430 can identify spans or regions of local similarity within a set or cluster of proteins.

The Structure Motif Module 410 identifies a set of structurally conserved residues that form a motif set based on a set of spans or one-to-one residue correspondences based on aligned protein structures and residue conservation. In one embodiment, the Structure Motif Module 410 is adapted to receive a set of spans from the Structure Pattern Detection Module 430.

In one embodiment, the Structure Motif Module 410 uses a set of one-to-one residue correspondences from aligned protein structures or “spans” to generate conservation scores for the protein residues in the correspondence. Residues in the one-to-one correspondence in a span are scored for conservation at each position in the span. Methods of generating residue conservation scores are discussed below in the section titled Conservation Uniqueness Scores.

In embodiments where the span includes the set of near-neighbor proteins, the Structure Motif Module 410 generates scores based on both conservation and uniqueness of the residues in the span correspondence. Methods of generating a conservation-uniqueness score are discussed in detail below in the section titled Conservation Uniqueness Scores (see patent application titled Structure Based Analysis for Identification of Protein Signature: cuScore by Carol Zhou and Adam Zemla filed Apr. 16, 2007).

According to the embodiment, the Structure Motif Module 410 uses the scored residues in the span correspondence to identify the subset of residues in the span which form a motif based on a cutoff conservation score value. The Structure Motif Module 410 also identifies motifs based on scored residues by generation of a distribution of residue conservation scores and selection of a percentile of top scoring residues to determine motifs.

The Structure Motif Module 310 also functions to generate clusters of motifs using a number of different methods of clustering. The Structure Motif Module 310 combines motifs from a variety of different sources. In some embodiments the Structure Motif Module 310 may cluster motifs from Protein Sequence Databases 450 such as InterPro or Protein Structure Databases 460 such as SCOP, alone or in combination with motifs identified by the Structure Motif Analysis Engine 400.

In embodiments in which the Structure Motif Analysis Engine 400 is adapted to communicate with the Sequence Motif Analysis Engine 300, the Structure Motif Module 310 can cluster sequence motifs identified by the Sequence Motif Module 310 alone or in combination with other motifs. In such embodiments, the Structure Motif Module 310 may also identify motifs to cluster based on Sequence Activity Relationship information or functional sequence annotations generated by the Sequence Activity Relationship Module 330 and the Sequence Function Module 320 respectively.

The Structure Motif Module 310 functions to generate clusters of motifs using a number of different methods of clustering. In some embodiments, clustering is a two step process including the generation of a set of pair-wise similarity values between a set of motifs and the subsequent generation of a cluster representative of the motif similarities based on the set of pair-wise values.

The Structure Motif Module 410 generates similarity values based on protein sequence alignments, protein structure alignments, conventional similarity metrics, motif functional annotations or any combination thereof. The Structure Motif Module 310 generates pair-wise similarity values based on two proteins or on a protein and a representative protein structure or sequence such as a substitution matrix.

In some embodiments, the Structure Motif Module 410 may combine a structural alignment with a sequence alignment in order to evaluate both structural conservation and side-chain similarity. In one embodiment, the Structure Motif Module 410 generates pair-wise similarity values based on combining and comparing a multiple sequence alignment generated using a multiple sequence alignment algorithm such as ClustalW (cite) with a local global multiple protein structure alignment and clustering algorithm such as STRALCP. In another embodiment, the Structure Motif Module 410 generates pair-wise similarity values based on combining conservation scores with a local global protein structure alignment and clustering algorithm such as STRALCP. According to the embodiment, the Structure Motif Module 310 may generate pair-wise similarity values based on other algorithms that combine sequence and structure alignments such as the Match Augmentation (MA) algorithm (Chen et al., 2005).

The Structure Motif Module 410 can also use qualitative data such as motif functional annotation to calculate or weight similarity values. It is imaginable that many methods of generating pair-wise similarity values will be apparent to those skilled in the art.

The Structure Motif Module 410 generates clusters based on the pair-wise similarity values. According to the embodiment of the present invention, any type of clustering algorithms such as hierarchical clustering (Heller and Ghahramani, 2005) or agglomerative clustering (Franti et al., 2006) can be used.

The Structure Function Module 440 accesses functional annotations from the Protein Function Databases 470 at the structure and the domain levels. Any system of functional annotation of domain sequence or structural elements can be used by the Sequence Function Module 320 to annotate motifs with associated functional information.

A first level of annotation is at the protein sequence level. Systems of annotation such as the Gene Ontology (available at the Gene Ontology website), the Enzyme Classification (EC), and the Swiss-Prot protein knowledgebase provide a functional annotation at the protein level. Other annotations at the protein level may be specific to the class of protein (e.g., toxin, enzyme) or taxonomy of organism. Examples of databases of microbial pathogen protein sequence function include the VIDA database (available at the website of the University College of London) and the ARGO database of vancomycin and b-lactam antibiotic resistance genes. An example of a database specific to a class of proteins (e.g., toxins) is the Tox-Prot subset of the Swiss-Prot protein knowledgebase (available at the European Bioinformatics Institute website). A number of systems of annotation are discussed in detail with respect to MvirDB in (Zhou et al, NAR 2007) and MannDB (Zhou et al, BMC Bioinformatics 2006), hereby incorporated by reference. It is expected that other systems of functional annotation of protein sequences will be apparent to those skilled in the art.

A second level of annotation is at the protein domain level. Domain of sub-sequences or sub-structures of proteins have recognized functional properties. Databases such as the Structural Classification of Proteins (SCOP, available at the website of the Medical Research Council of Cambridge, UK) compiles sub-structures and sub-sequence associated with functional protein domains as well as the associated structural families of the proteins. Likewise, InterPro (available at the website of the European Bioinformatics Institute) is a database which contains protein families, functional sites and domains which may be applied to characterize unknown protein sequences. The PRINTS database of protein classifications contains motifs which confer protein functionality; and the virulence subset of PRINTS contains motifs conferring functionality in different mechanisms of virulence. The Structure Function Module 320 associates a structural motif with a functional annotation of the domain in which it occurs.

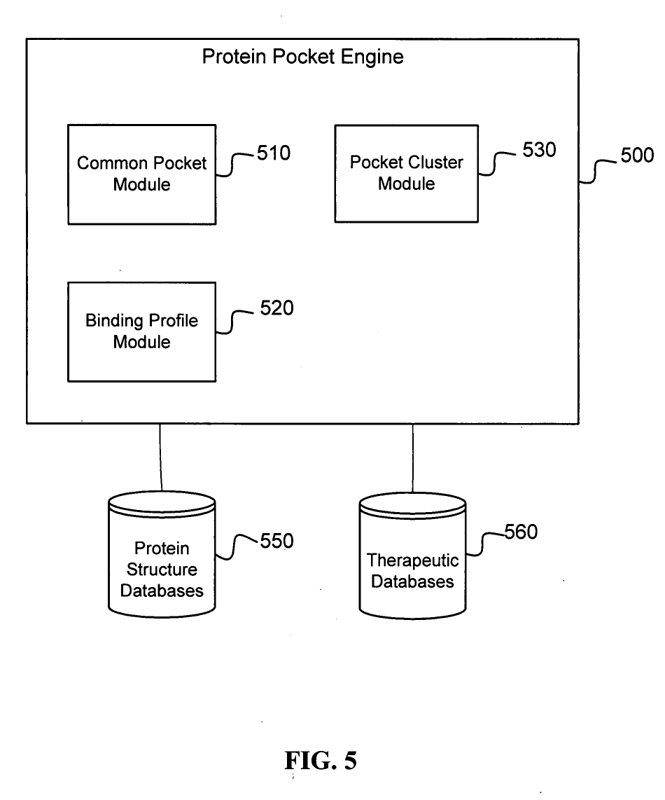

FIG. 5. is a block diagram illustrating the Protein Pocket Engine 500 according to one embodiment. The Protein Pocket Engine 500 is comprised of three modules, a Common Pocket Module 510, a Pocket Binding Module 530 and a Pocket Cluster Module 530.

The Common Pocket Module 510 identifies sets of residues which form three-dimensional concavities or “pockets” on the surface of a protein structure. According to the embodiment of the present invention, the Common Pocket Module receives one or more protein structures as input or imports the protein structures from protein structure databases. In some embodiments, the Pocket Binding Module is adapted to communicate directly with the Therapeutic Databases 560 and the Protein Structure Databases 550.

In one embodiment, the Common Pocket Module 510 receives a set of residues which form a conserved motif in a set of homologous proteins from the Structure Motif Analysis Engine 400 or the Sequence Motif Analysis Engine 300. The Common Pocket Module 510 identifies a set of conserved or “common pockets” based on the set of residues which form a conserved motif and reference protein structure using a method of conserved protein pocket identification. In one embodiment, the Common Pocket Module uses Common Pocket as a method of conserved protein pocket identification. Common Pocket is discussed in detail in the section below titled Protein Pocket Identification.

In other embodiments, the Common Pocket Module identifies protein pockets based only on protein structure. According to the embodiment, pockets may be identified based on a single protein structure or multiple protein structures. These methods are discussed in detail below in the section titled Protein Pocket Identification.

The Pocket Binding Module 530 uses computational docking methods to virtually screen an identified protein pocket with a series of small molecules or “ligands”. According to the embodiment of the present invention, the Pocket Binding Module 530 accesses the Therapeutic Databases 560 such as DrugBank to import molecular structures of ligands or small molecules. The Pocket Binding Module 530 uses methods of computational protein ligand docking to evaluate binding of a set of ligands or small molecules to a pocket. Suitable computational docking algorithms and scoring functions are discussed below in the section titled Protein Ligand Docking. The Pocket Binding Module 530 generates a profile of all small molecules that bind to each pocket, enabling the characterization of the pocket by its binding profile.

The Pocket Cluster Module 530 clusters the binding profiles. The Pocket Cluster Module generates two types of clusters: clusters of motifs with similar binding profiles and clusters of molecules that bind to similar motifs. In some embodiments, the creation of clusters is a two step process of generating pair-wise similarity values between motifs or clusters and generating clusters based on the pair-wise similarity values.

The Pocket Cluster Module 530 generates pair-wise similarity values according to a number of different methods. In one embodiment, the binding profiles of two motifs or molecules are represented as binary vectors and the Pocket Cluster Module 530 generates pair-wise similarity values by scoring the number of shared binding reactions between the two motifs or molecules. The Pocket Cluster Module 530 further generates representative binding profiles by combining the binding profiles of a set of pockets associated with a protein family, a group of organisms or any other user specified set of pockets.

The Pocket Cluster Module 530 generates pair-wise similarity values between on binding profiles for two different pockets. The Pocket Cluster Module 530 also generates pair-wise similarity values based on a binding profile for a pocket and a representative binding profile. In another embodiment, pair-wise similarity values are based on correlation between the two vectors representing two motifs. Other methods of calculating pair-wise similarity values will be apparent to those skilled in the art.

The Pocket Cluster Module 530 generates clusters of motifs and molecules based on the pair-wise similarity values. The pair-wise similarity value may be clustered using any kind of algorithm such as hierarchical clustering, agglomerative clustering or k-means clustering (Bottegoni, et al., 2006). Other suitable clustering algorithms will be apparent to those skilled in the art.

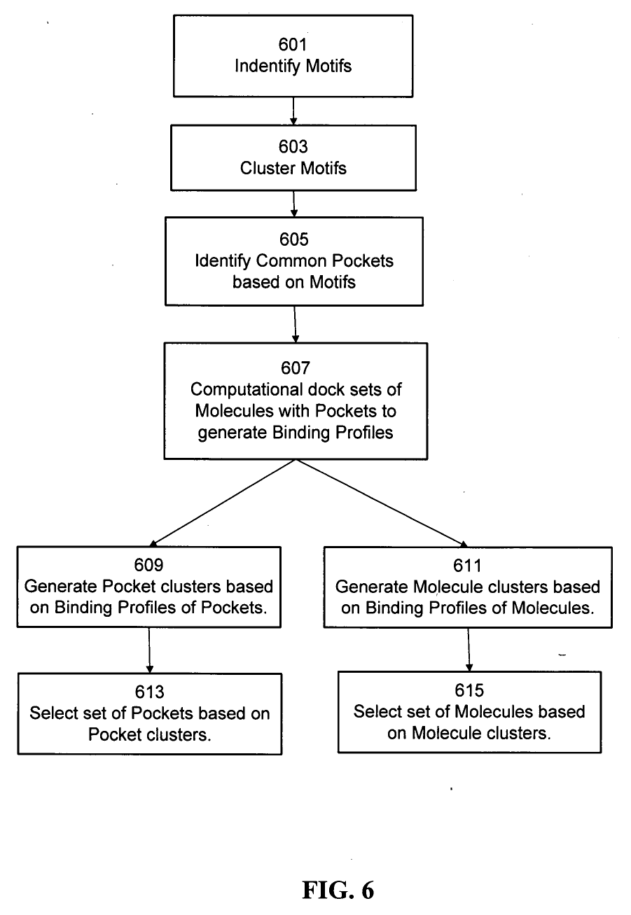

FIG. 6. is a flow chart illustrating the high level steps of target motif and molecule identification according to one embodiment. Subsets of target three-dimensional concavities and molecules are identified from a set of motifs.

Initially, the Structure Motif Module 410 identifies a set of motifs 601. The Structure Motif Module 410 identifies 601 structural motifs generated by the Structure Motif Module 410. The Structure Motif Module 410 also identifies 601 sequence motifs generated by the Sequence Motif Module 310. The Structure Motif Module further identifies sequence and structure motifs based on Sequence Databases and Structure Databases.

The Structure Motif Module 410 clusters 603 the identified motifs. The Structure Motif Module 410 generates pair-wise similarity values 603 based on protein sequence alignments, protein structure alignments, conventional similarity metrics, motif functional annotations or any combination thereof. The Structure Motif Module 410 generates clusters 603 based on the pair-wise similarity values.

The Common Pocket Module 510 identifies pockets 605 or three dimensional cavities on the surface of a protein structure based on the motifs. The Common Pocket Module 510 identifies conserved residues in a motif that form pockets 605 in a reference protein structure using the Common Pocket algorithm (discussed below in the section titled Protein Pocket Identification).

The Binding Profile Module 520 generates binding profiles 607 by evaluating binding of a set of ligands or small molecules to a pocket. Using computation protein ligand docking methods, The Pocket Binding Module 530 generates binding profiles 607 describing the set of molecules that bind to each pocket, enabling the characterization of the pocket and molecules by their binding profiles.

The Pocket Cluster Module 530 generates 609 pair-wise similarity values based on the binding profiles of the pockets. The Pocket Cluster Module 530 generates clusters 609 based on the binding profiles of the pockets. A group or set of pockets is selected for targeted broad-spectrum drug development 613 based on the pocket clusters.

The Pocket Cluster Module 530 also generates 611 pair-wise similarity values based on the binding profiles of the pockets. The Pocket Cluster Module 530 generates 611 molecule clusters based on the binding profiles of the molecules. A group or set of molecules is selected for targeted broad-spectrum drug development 613 based on the molecule clusters.

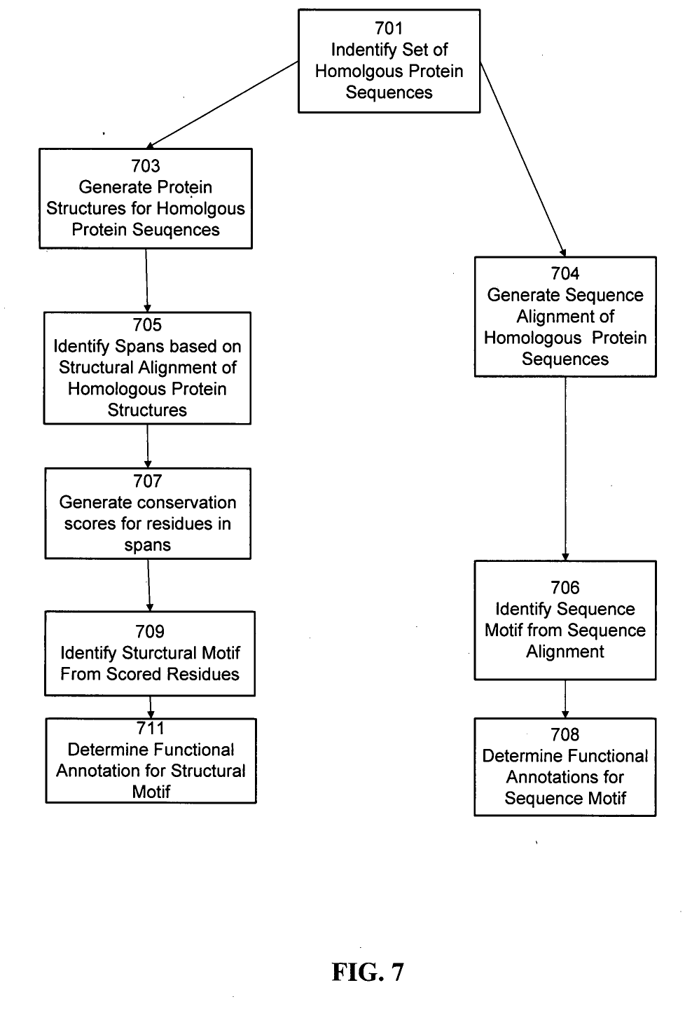

FIG. 7. is a flow chart illustrating the high level steps of sequence and structure motif generation according to one embodiment.

Initially, the Protein Set Selection Engine 200 identifies 701 a set of homologous proteins. The Proteome-Proteome Correlation Module 205 identifies 701 a set of homologous proteins for a protein or sets of proteins within proteomes of a set of specified organisms. The Protein Set Selection Module 215 allows the selection of a group of known homologs based on taxonomy or other criteria.

In structure motif generation, the Sequence to Structure Module 420 generates 793 protein structures for proteins in the set of homologous proteins with unsolved protein structure. The Sequence to Structure Module 420 generates 793 protein structure using ab-initio modeling, structure-sequence based methods of protein structure modeling and sequence-sequence based methods of protein structure modeling.

The Structure Pattern Detection Module 430 generates spans 705 or one-to-one correspondences between residues in the aligned protein based on the structural alignment of protein structures. Based on the generated and known protein structures of the homologous set of proteins, the Structure Pattern Detection Module 430 generates a structural alignment 705. The Structure Pattern Detection Module 430 generates structural alignments 705 based on local structure alignment, global structure alignment or any combination thereof. The Structure Pattern Detection Module 430 generates spans 705 using STRALCP (Zemla et. al, in preparation, see Provisional Application No. 60/813,211).

The Structure Motif Module 410 generates conservation scores 707 for residues within the spans. The Structure Motif Module 410 generates conservation scores 707 at each position in the one-to-one correspondence of the span using similarity metrics. Methods of generating conservation scores 707 are discussed below in the sections titled Conservation Uniqueness Scores and Similarity Metrics.

The Structure Motif Module 410 identifies motifs 709 based on the conservation scores. The Structure Motif Module 410 identifies 709 motifs based on a user-specified threshold conservation score value. The Structure Motif Module 410 also identifies 709 motifs based on a threshold value determined from a distribution of conservation score values.

The Structure Function Module 440 determines 711 functional annotations of the motifs. The Structure Function Module 440 is adapted to communicate with Protein Function Databases 470 to determine 711 annotations of functional properties associated with one or more residues comprising a protein structure motif.

In sequence motif generation, the Sequence Motif Module 310 generates 704 a sequence alignment of the homologous protein sequences. The Sequence Motif Module 310 generates 704 multiple sequence alignments, pair-wise sequence alignments, distance matrices and probabilistic models.

The Sequence Motif Module 310 identifies 706 sequence motifs. The Sequence Motif Module 310 identifies 706 sequence motifs based generating similarity metrics based on aligned sequences. The Sequence Motif Module 310 also identifies 706 sequence motifs based on probabilistic models.

The Sequence Function Module 320 determines 708 functional annotations of the sequence motifs. The Sequence Function Module 320 is adapted to communicate with Therapeutic Databases 360 to determine 708 annotations of drug binding activities associated with one or more residues in a protein sequence motif. The Sequence Function Module 320 is also adapted to communicate with Protein Function Databases 350 to determine 708 annotations of functional properties associated with one or more residues in the protein sequence motif.

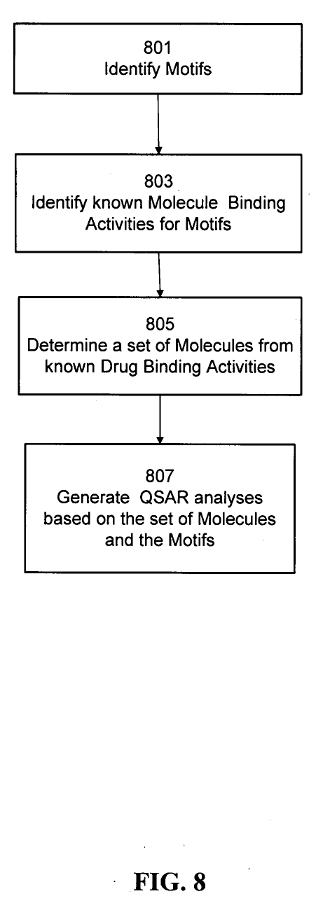

FIG. 8 is a flow chart illustrating the high level steps of motif quantitative structure activity relationship (QSAR) determination according to one embodiment.

Initially, the Structure Motif Module 410 identifies a set of motifs 801. The Structure Motif Module 410 identifies 801 structural motifs generated by the Structure Motif Module 410. The Structure Motif Module 410 also identifies 801 sequence motifs generated by the Sequence Motif Module 310. The Structure Motif Module further identifies sequence and structure motifs based on Sequence Databases and Structure Databases.

The Sequence Function Module 320 identifies 803 a set of molecules with known binding activity to protein motif sequence. The Sequence Function Module 320 is adapted to communicate with Therapeutic Databases 360 to identify 708 annotations of molecules with known drug binding activity to be associated with one or more residues in a protein sequence motif.

The Sequence Activity Relationship Module 330 determines 805 an expanded set of molecules based on the set of molecules with known binding activity to protein motif sequence. The Sequence Activity Relationship Module 330 determines 805 a set molecules from MDDR-3D with significant similarity to the set of molecules with known binding activity to protein motif sequence from BRENDA and DrugBank based on analysis of chemical similarity between the molecules in the databases. The Sequence Activity Relationship Module 330 determines 805 chemical similarity between molecules using a signature descriptor (Faulon, 2004) to represent molecule data as feature vectors in order to facilitate similarity analyses.

The Sequence Activity Module 330 generates 807 Quantitative Structure Activity Relationship (QSAR) analyses to predict binding of the sequence motifs to the expanded set of molecules. The Sequence Activity Module 330 generates 807 the QSAR analysis based on a signature descriptor (Faulon, 2004).

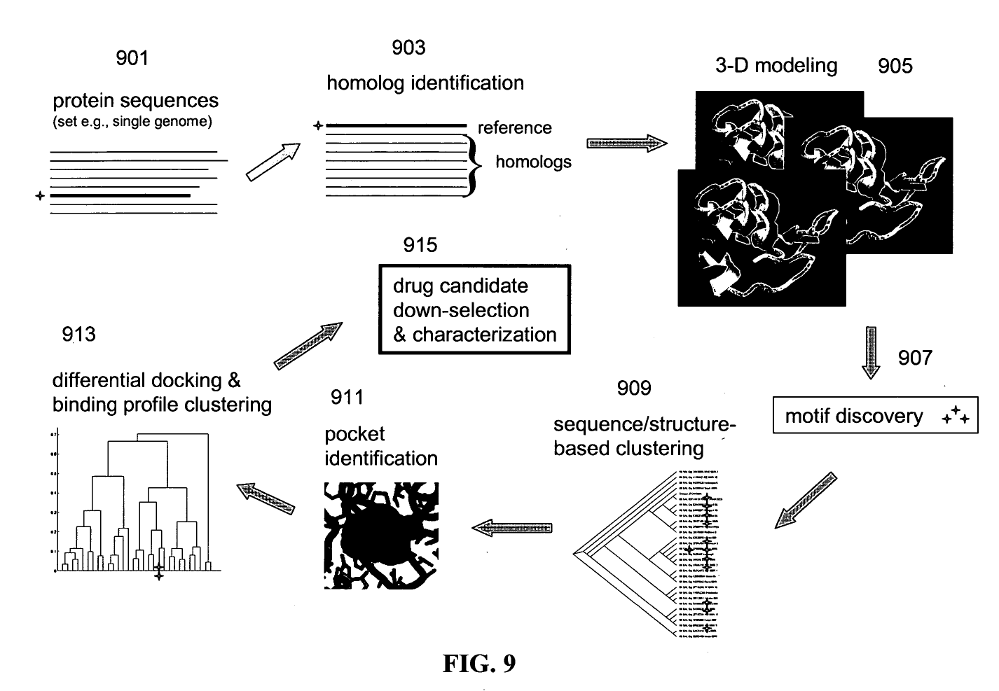

FIG. 9 illustrates the steps performed in the working example of the system. Initially, a target sequence and/or organisms are identified 902 from a set of sequences. In the working example, the target organisms identified 902 were from the family Flaviviridae. This family includes viruses responsible form many human encephalitic and hemorrhagic diseases such as Yellow Fever, West Nile, Dengue, Japanese and tick-borne encephalitis. There are a number of resources available to study the sequence. Flaviviruses consist of a single stranded RNA, three structural proteins, and seven non-structural proteins. For the working example, serine protease one of the non-structural proteins was identified 902 as a target protein. The protein structure of serine protease has been empirically solved (crystallized) for West Nile virus (PDB ids: 2IJO, 2GGV, 2FP7) and for Dengue virus (2FOM).

A set of homologous proteins for the target organisms and sequence were identified 903. In the working example, 30 known Flaviviridae serine protease sequences were selected 903 as a working set of homologous proteins. Resources available to study the sequence similarity of Flaviviridae include Flavitrack (available at the website of the University of Texas Medical Branch).

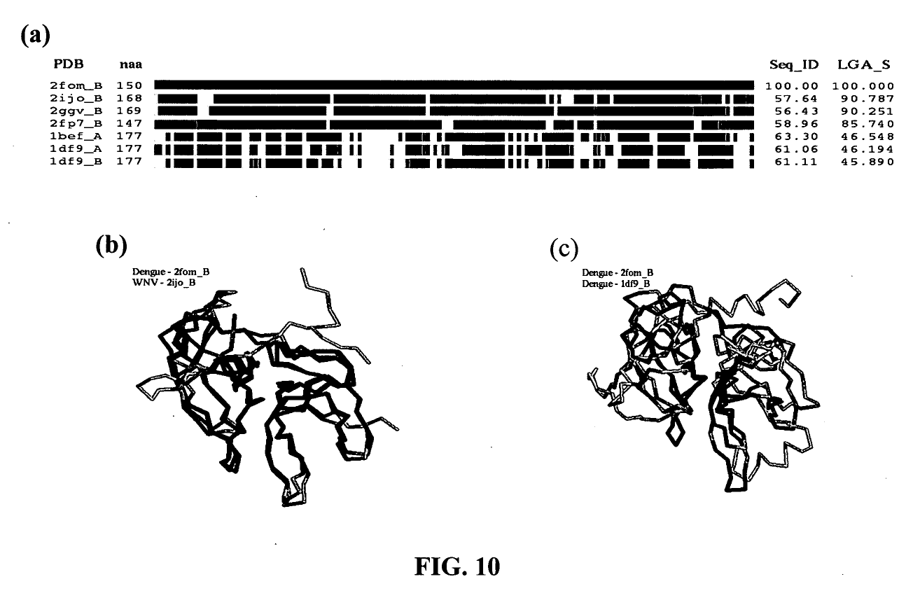

Three dimensional models were generated 905 based on the identified set of protein homologs. In the working example, the AS2TS system (cite) was used to generate three dimensional models based on identifying empirically solved crystal Flavivirus serine protease structures in Protein Data Bank (PDB) and using the empirically solved crystal structures as templates for the modeling process. Structural analysis of the identified homologs showed that some crystal structures from Dengue (1BEF, and 1DF9 chain A and chain B) were to be removed from the analysis due to their significant conformation changes (see FIG. 2) upon activation and binding an inhibitor. Structural analysis of the identified homologs is discussed in detail with respect to FIGS. 10(a), 10(b) and 10(c).

Motifs were discovered 907 based on the generated and known set of three dimensional protein structures. In the working example, LGA was first used to generate 907 a multiple sequence alignment of all the protein structures. This step is discussed further with respect to FIG. 1.

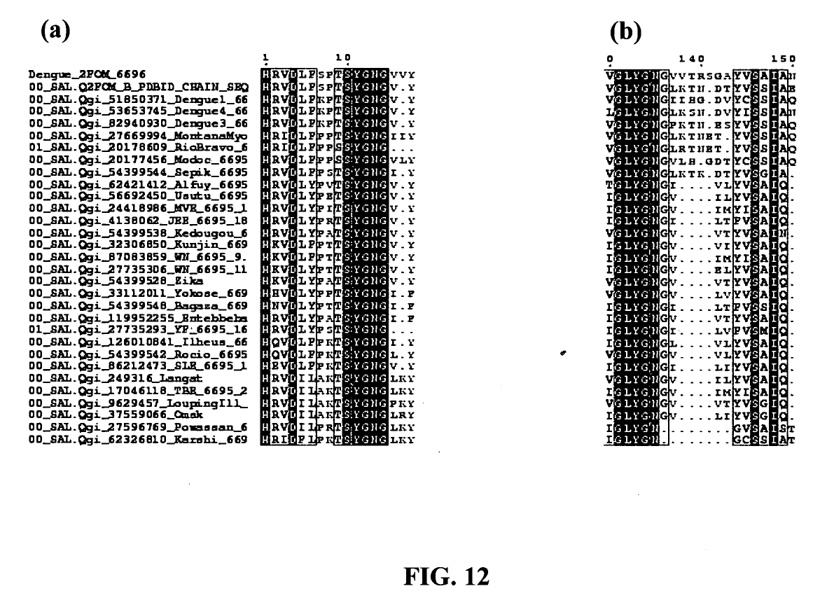

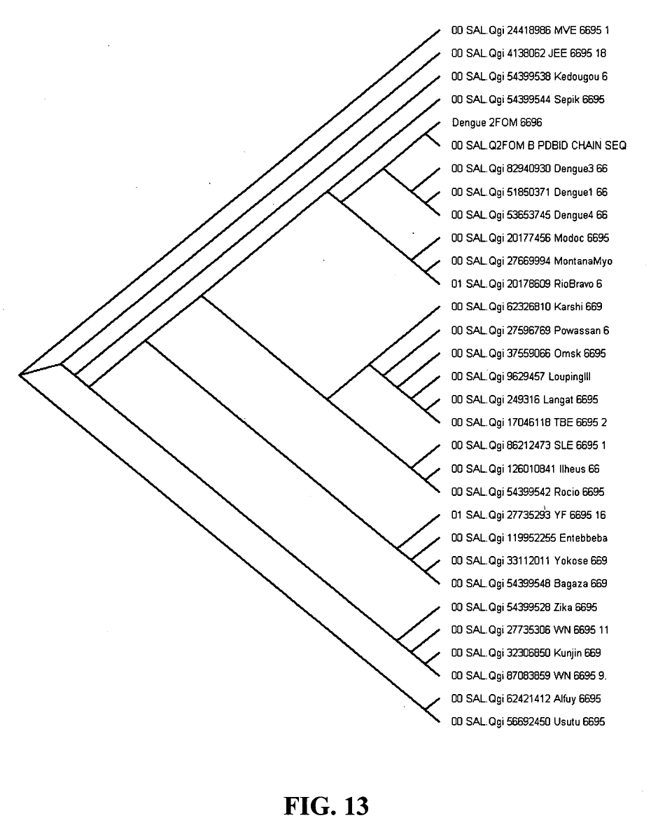

Sequence and structure based clustering 909 was performed to identify a subset of motifs for pocket identification. STRALCP was used to generate 909 a structural alignment of the set of protein structures for clustering. ClustalW was used to generate 909 a multiple sequence alignment for clustering. These steps are discussed further with respect to FIGS. 12(a) and 12(b). A dendogram was created by clustering 909 the Flaviviruses by motifs. This dendogram is discussed with respect to FIG. 13.

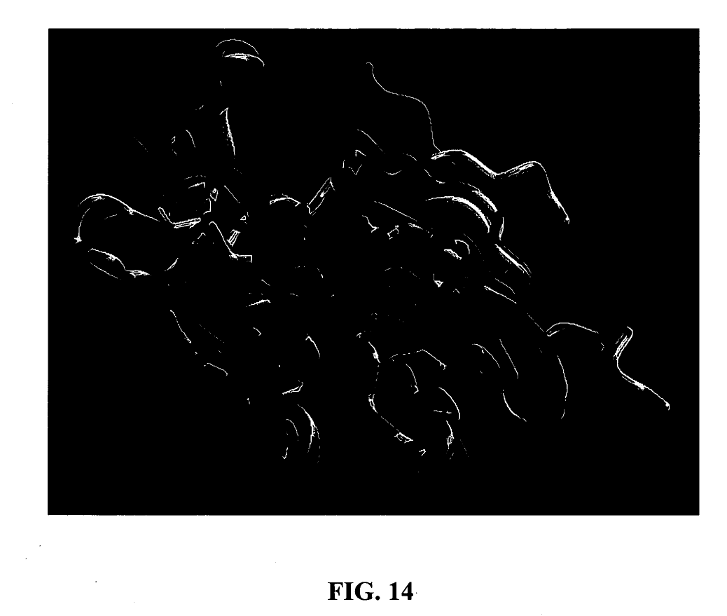

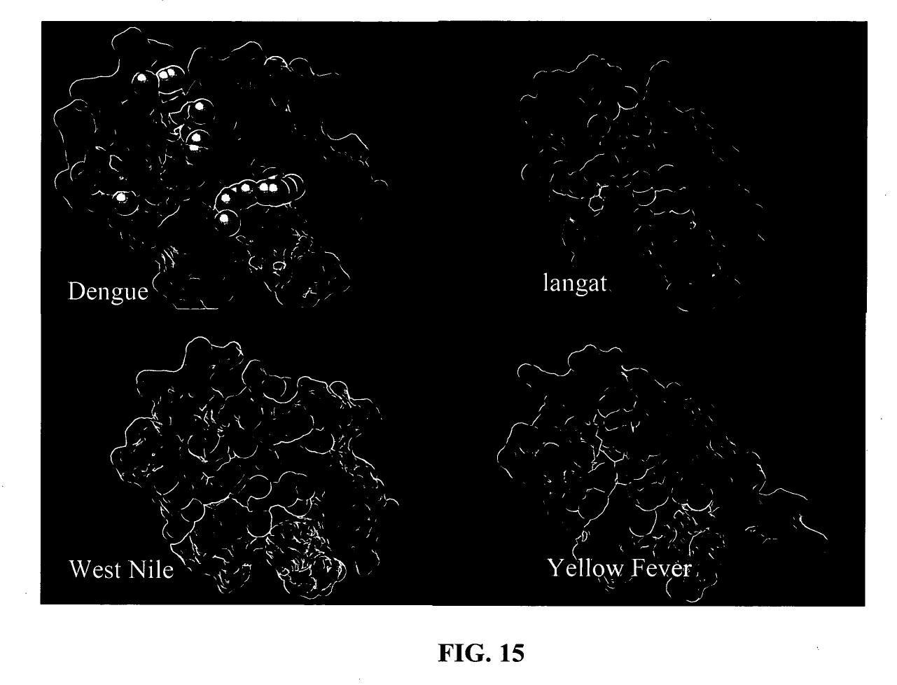

Protein pockets were identified 911 based on a subset of motifs and corresponding protein structures. The subset of motifs and protein structures were identified from the dendogram created from the sequence and structure based clustering. In the working example, four representative Flavivirus motifs and protein structures for West Nile virus, Langat virus, Dengue Virus and Yellow Fever virus were selected based on the dendogram shown in FIG. 13. Pockets in the representative protein models were identified 911 using Common Pocket. Pocket identification is further discussed with respect to FIG. 14 and FIG. 15.

Binding profiles were generated 913 and clustered 913 using computational differential docking of small molecules. A test database consisting of approximately 1100 diverse compounds was docked 913 into each of the 30 structural models, using the UCSF DOCK6.0 program. Common pockets identified in each of the four representative protein structures (FIG. 15) were used by the docking program to orient each small molecule by matching sphere centers to ligand centers as described in the DOCK program (Kuntz, 1982), and treating common and uncommon spheres the same.

As a control, four sets of spheres corresponding to each of the four representative pockets identified using Common Pocket were docked into each of the 30 structures. The exact choice of the Common Pocket sphere set did not alter the docking scores since their function in this docking run was to provide a sufficient number of ligand orientations to sample the pocket space. The common sub-sites within each sphere set can be used in subsequent docking calculations to only consider ligands that bind to common regions and exclude uncommon regions, which will affect the docking scores depending on the shape and location of the common sphere regions.

FIG. 10(a) is a graphic illustration of the pair-wise structural alignment of 7 homologous proteins with serine protease from Dengue virus (PDB code: 2fom_B) using the Local Global Alignment (LGA) program. Colored bars represent distance deviation of the alpha carbons between superimposed PDB structures and Dengue virus (PDB code: 2fom_B). The alignment is 150 residues in length from the left (N terminal) to the right (C terminal)). Residues superimposed with a distance between alpha carbons below 2 Å are represented in green. Distances below 4 Å are represented in yellow. Distances below 6 Å are represented in orange. Residues with distance between alpha carbons at or above 6.0 Å are represented in red. Unaligned terminal residues are represented in grey.

FIG. 10(b) illustrates 3D representation of the superimposed structures of Dengue virus serine protease (PDB code: 2fom_B) and West Nile Virus serine protease (PDB code: 2ijo_B). The superpostion corresponds to structural alignment represented in the second to top colored bar in FIG. 10(a). The coloring of the residues is correspondent to FIG. 10(a).

FIG. 10(c) 3D representation of two superimposed structures of Dengue virus serine protease (2fom_B and 1df9). This plot shows significant conformation changes between two serine proteases from Dengue due to activation and binding. The superpostion corresponds to structural alignment represented in the bottom colored bar in FIG. 10(a). The coloring of the residues is correspondent to FIG. 10(a).

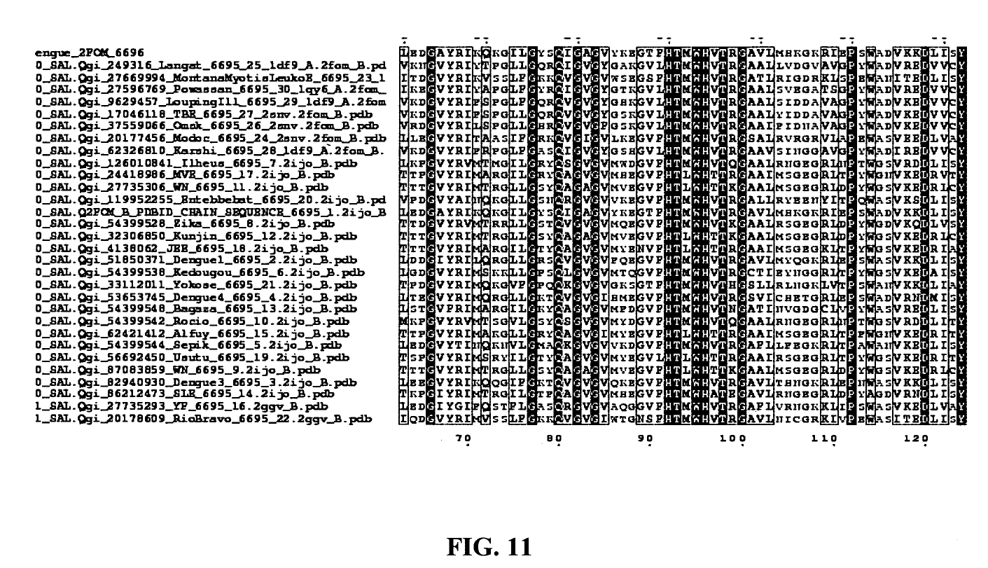

FIG. 11 is a graphic illustration of a portion of a multiple structure alignment between the protein structure of serine protease of the Dengue virus and 30 homologous protein structures. Orange colored residues in the alignment represent residues that are structurally conserved in all homologous protein structures. Yellow colored residues represent residues that are highly conserved in the homologous protein structures

FIG. 12(a) is a graphic illustration of a structure alignment of a structural motif identified based on the alignment illustrated in FIG. 11 and identification of active binding sites using Common Pocket. FIG. 12(b) is a graphic illustration of a sequence alignment generated based on the sequences associated with protein sequences associated with the structural protein motif in FIG. 12(a).

FIG. 13 is a dendogram created by clustering the flavivirus sequences based on sequence and structural motif alignments illustrated in FIGS. 12(a) and 12(b).

FIG. 14 is a “ribbon diagram” of the 4 representative flavivirus protease structures, each taken from a separate cluster based on the structure/sequence/motif alignment. The common motif is highlighted in green in all structures. The Langat protein structure is represented in magenta. West Nile virus and Dengue viruses are represented in cyan. Yellow fever virus is represented in yellow. In this example, the West Nile and Dengue are modelled from same template so have the same backbone structure, but sidechains and overall shapes of the protein structures of the two viruses differ.

FIG. 15 illustrates the pockets identified from the representative protein structures of the Dengue, Langat, West Nile and Yellow Fever viruses. The proteins are shown with a solvent-excluded surface colored in white with red representing the catalytic triad. Pockets are shown with spheres in each of the representative structure in cyan and green. The green spheres are the common sub-sites within the pocket for this cluster.

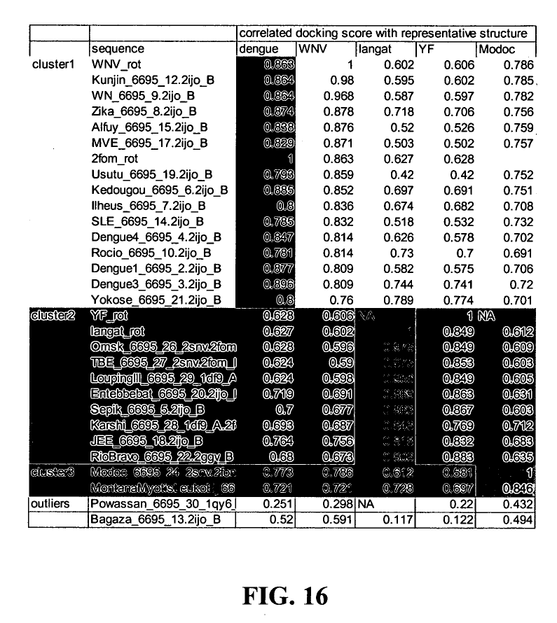

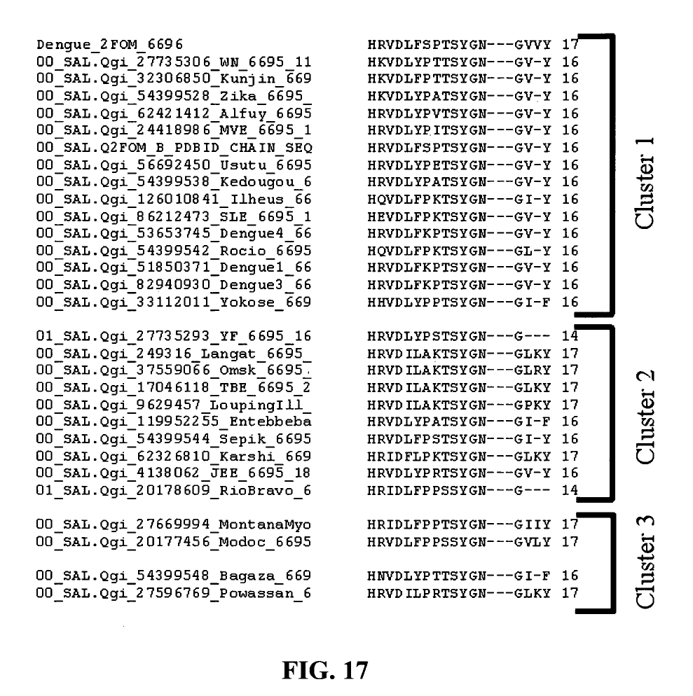

FIG. 16 is a graphic illustration of the protein motif clusters generated for the set of homologous proteins based on binding profiles. The left most column denotes the distinct clusters based on binding profiles. The sequence column contains the sequence identifier of the protein structures. The third, fourth, fifth and sixth columns contain correlation values between each protein structure to representative dock scores generated for Dengue virus, West Nile Virus, Langat virus, and Yellow Fever virus, respectively. Modac virus was added to the set of 4 representative structures for a total of 5. Correlations of the representative structure with itself were always 1. The results were clustered using a cutoff of 0.8 for structures to be considered in the same cluster. There were two main clusters of dock scores, and cluster 3 just misses the cutoff to be in cluster 1. Cluster 1 comprises compounds with binding profiles correlated to Dengue, although they also correlate to WNV which is in that cluster. Cluster 2 comprises compounds with binding profiles correlated with Langat. Cluster 3 correlates with Modoc virus.

Reference in the specification to “one embodiment” or to “an embodiment” means that a particular feature, structure, or characteristic described in connection with the embodiments are included in at least one embodiment of the invention. The appearances of the phrase “in one embodiment” in various places in the specification are not necessarily all referring to the same embodiment.

Some portions of the above are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “determining” or the like, refer to the action and processes of a computer system, or similar electronic computing device, that manipulates and transforms data represented as physical (electronic) quantities within the computer system memories or registers or other such information storage, transmission or display devices.

Certain aspects of the present invention include process steps and instructions described herein in the form of an algorithm. It should be noted that the process steps and instructions of the present invention can be embodied in software, firmware or hardware, and when embodied in software, can be downloaded to reside on and be operated from different platforms used by a variety of operating systems.

The present invention also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and displays presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein, and any references below to specific languages are provided for disclosure of enablement and best mode of the present invention.

While the invention has been particularly shown and described with reference to a preferred embodiment and several alternate embodiments, it will be understood by persons skilled in the relevant art that various changes in form and details can be made therein without departing from the spirit and scope of the invention.

Finally, it should be noted that the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the following claims.

Protein Pocket Identification

Three dimensional concavities or crevices on the surface of a protein structure can confer ligand binding capabilities on an organism. Computational models seek to identify these concavities or “pockets” by characterization of the three dimensional structure of the protein.

Some methods of protein pocket identification use triangulation such as weighted Delaunay triangulation to determine pocket volumes (Liang et al., 1998). Other methods use spheres to determining protein pocket volumes (Laurie and Jackson, 2005).

Conserved protein pocket identification seeks to identify conserved pockets through associating the residues that form pockets with residues which form a conserved motif in homologous protein sequences or structures.

One method of identifying conserved protein pockets entails filling the three dimensional protein structures with spheres, creating a “negative image” of the structure. A cutoff distance, such as 8 Angstroms, is used to determine spheres which are proximate to conserved residues. Spheres are labeled as conserved or not-conserved based on their proximity to residues that form a conserved motif. The conserved spheres are clustered based on their three dimensional coordinates to identify a set of spheres that interact with conserved residues and are proximal in three dimensional space forming a cluster. This method can also be applied to identify pockets with residues that are unique to a protein (Zhou, Bioinfomatics 2005).

Similarity Metrics

Various similarity metrics are used to score the uniqueness or conservation of the residues in a correspondence. These metrics include but are not limited to a trinary system or substitutions matrices. It is expected that those skilled in the art can envision a variety of comparable similarity metrics for calculating conservation and uniqueness.

In one embodiment of the present invention, the similarity metric is based on a trinary system of residue identity, non-identity and similarity. Residues from each sequence in a correspondence are compared with the corresponding residue in the reference protein. Alternately, residues from each sequence are compared with a consensus residue identified in the majority of the sequences in set of the correspondences. Residue identity refers to the residue comprising the same amino acid as the residue to which it is compared. Residue similarity refers to the two residues under comparison being part of a pre-defined group or family with similar features. If two residues are neither identical nor similar, the residues are non-identical. Scores of 1, 0 and 0.5 are assigned based on identity, non-identity and similarity respectively. It is expected that those skilled in the art can imagine a variety of different scoring techniques.

Various pre-defined groupings used to specify may be employed in this technique. Amino acids are referred to herein by corresponding single letter symbols as defined by IUPAC (International Union of Pure and Applied Chemistry), a table listing amino acids and their corresponding single letter symbols may be found in a standard biochemistry textbook, for example, Leningher, Principles of Biochemistry, WH Freeman & Co (2004). One method of grouping the 20 known amino acids is by chemistry and size: aliphatic (AGILPV), aromatic (FWY), acidic (DE), basic (RKH), small hydroxylic (ST), sulfur-containing (CM) and amidic (NQ).

Other grouping schemes are based on functional properties such as: acidic (DE); basic (RKH); hydrophobic non polar (AILMFPWV); and polar uncharged (NCQGSTY). An example of a grouping scheme based on the charge of amino acid is: acidic (DE); basic (RKH) and neutral (AILMFPWV NCQGSTY). A grouping scheme based on structural properties of amino acids is: ambivalent (ACGPSTWY); external (RNDQEHK); internal (ILMFV) (Karlin and Ghandour, 1985). Other grouping schemes based on physical properties such as codon degeneracy or kinetic properties can also be employed.

In an alternate embodiment, substitution matrices may be used to calculate the similarity metric. Substitution matrices represent to the rate at which each possible residue in a sequence changes to each other residue over time. Substitution matrices are 20 by 20 matrices containing preferred substitutions propensity for all possible pairs of amino acids. The preferred substitution propensities may be calculated based on a set of homologous sequences or many sets of homologous sequences. Two substitution matrices for amino acids commonly used in the art are PAM (Point Accepted Mutation) and BLOSUM (BLOck SUbstitution Matrix). Substitution matrices may also be used to create a grouping such as above by identifying the grouping of amino acids which minimizes the off diagonal elements in the substitution matrix (Fygenson et al., 2004).

Protein Structure Alignment

Protein structure alignments preferably are sets of correspondences between spatial coordinates of sets of carbon alpha atoms which form the ‘backbone’ of the three-dimensional structure of polypeptides, although alignments of other backbone or side chain atoms also can be envisioned. These correspondences are generated by computationally aligning or superimposing two sets of atoms in order to minimize distance between the two sets of carbon alpha atoms. The root mean square deviation (RMSD) of all the corresponding carbon alpha atoms in the backbone is commonly used as a quantitative measure of the quality of alignment. Another quantitative measure of alignment is the number of equivalent or structurally aligned residues.

A variety of methods for generating an optimal set of correspondences can be used in the present invention. Some methods use the calculation of distance matrices to generate an optimal alignment. Other methods maximize the number of equivalent residues while RMSD is kept close to a constant value.

In the calculation of correspondences, various cutoff values can be specified to increase or decrease the stringency of the alignment. These cutoffs can be specified using distance in Angstroms. Depending on the level of stringency employed in the present invention, the distance cutoff used is less than 10 Angstroms or less than 5 Angstroms, or less than 4 Angstroms, or less than 3 Angstroms. One of ordinary skill will recognize that the utility of stringency criterion depends on the resolution of the structure determination.

In another embodiment of the present invention, the set of residue-residue correspondences are created using a local-global alignment (LGA), as described in US Patent Application Number 2004/0185486. In this method, a set of local superpositions are created in order to detect regions which are most similar. The LGA scoring function has two components, LCS (longest continuous segments) and GDT (global distance test), established for the detection of regions of local and global structure similarities between proteins. In comparing two protein structures, the LCS procedure is able to localize and superimpose the longest segments of residues that can fit under a selected RMSD cutoff. The GDT algorithm is designed to complement evaluations made with LCS searching for the largest (not necessary continuous) set of ‘equivalent’ residues that deviate by no more than a specified distance cutoff.

Quantitative Structure-Activity Relationship Modeling

Quantitative Structure-Activity Relationships (QSAR) are quantitative models in which chemical structure of a protein is quantitative correlated with processes such as biological activity and chemical reactivity. In QSAR models, activity is defined numerically by quantitative physiochemical properties and structural properties.

Two dimensional QSAR uses three dimensional protein data represented in 2 dimensions as molecular descriptors. Molecular descriptors used in 2D-QSAR include steric parameters, electronic shape parameters, hydrophobicity parameters and topological descriptors (Yang and Huang, 2006).

One method of encoding three dimensional molecular information as features in two dimensional vector space is by use of a signature descriptor (Faulon, 2004). In this method, the signature of an atom in a molecule is a canonical representation of the atom's environment up to a predefined height h. The signature of a molecule is a vector of occurrence numbers of atomic signatures. Signatures can then be trained to predict chemical reactivity through methods of machine learning such as support vector machines (SVMs).

Three dimensional QSAR (3D-QSAR) uses force field calculations which require coordinates of a three dimensional protein structure. One method of 3D-QSAR is Comparative Molecular Field Analysis (CoMFA, Cramer et. al 1988). Other methods of 3D-QSAR incorporate comparisons of different sets of molecular descriptors such as Comparative Molecular Similarity Indices Analysis (CoMSIA, Klebe et al. 1994).

Protein Ligand Docking

Protein-ligand docking aims to employ principles by which protein receptors recognize, interact, and associate with molecular substrates and inhibitors to predict the structure arising from the association between a given ligand and a target protein of known 3D structure (Sousa, 2006).

In protein-ligand docking, the search algorithm should allow the degrees of freedom of the protein-ligand system to be sampled sufficiently as to include the true binding modes. Three general categories of algorithms have been developed to address this problem of ligand flexibility: systematic methods; random or stochastic methods; and simulation methods.

Systematic search algorithms attempt to explore all degrees of freedom in a molecule. These algorithms can be further divided into three types: conformational search methods, fragmentation methods, and database methods.

In conformational search methods, all rotatable bonds in the ligand are systematically rotated through 360° using a fixed increment, until all possible combinations have been generated and evaluated. As the number of structures generated increases immensely with the number of rotatable bonds (combinatorial explosion), the application of this type of method, in its purest form, is very limited.

Fragmentation methods use two different approaches to incrementally grow the ligands into the active site. One approach is by docking the several fragments into the active-site and linking them covalently to recreate the initial ligand (“the place-and-join approach”). Another approach is by dividing the ligand into a rigid core-fragment that is docked in first place and flexible regions that are subsequently and successively added (“the incremental approach”). DOCK (Kuntz, 1982) is an example of a docking programs that use a fragmentation search method.

Database methods have been devised using libraries of pre-generated conformations or conformational ensembles to address the combinatorial explosion problem. A example of a docking program using database methods is FLOG (Miller et al., 1994), which generates a small set of 25 database conformations per molecule based on distance geometry, which are subsequently subject to a rigid docking protocol.

Random search algorithms sample the conformational space by performing random changes to a single ligand or a population of ligands. At each step, the alteration performed is accepted or rejected based on a predefined probability function. There are three basic types of methods based on random algorithms: Monte Carlo methods (MC), Genetic Algorithm methods (Yan and Liu, 2006), and Tabu Search (Glover and Laguna, 1998) methods.

Simulation methods employ a rather different approach to the docking problem, based on the calculation of the solutions to Newton's equations of motion. Two major types exist: molecular dynamics (MD) and pure energy minimization methods.

Scoring functions normally employed in protein-ligand docking are generally able to predict binding free energies within 7-10 kJ/mol and can be divided into three major classes: force field-based, empirical, and knowledge-based scoring functions.

In force-field based scoring, standard force fields quantify the sum of two energies: the interaction energy between the receptor and the ligand, and the internal energy of the ligand. The energies are normally accounted through a combination of a van der Waals with electrostatic energy terms. A Lennard-Jones potential is used to describe the van der Waals energy term, whereas the electrostatic term is given by a Coulombic formulation with a distance-dependent dielectric function that lessens the contribution from charge-charge interactions.

Empirical scoring functions are based on the idea that binding energies can be approximated by a sum of several individual uncorrelated terms. Experimentally determined binding energies and sometimes a training set of experimentally resolved receptor-ligand complexes are used to determine the coefficients for the various terms by means of a regression analysis.

Knowledge-based scoring functions focus on following the rules and general principles statistically derived that aim to reproduce experimentally determined structures, instead of binding energies, trying to implicitly capture binding effects that are difficult to model explicitly. Typically, these methods use very simple atomic interactions-pair potentials, allowing large compound databases to be efficiently screened. These potentials are based on the frequency of occurrence of different atom-atom pair contacts and other typical interactions in large datasets of protein-ligand complexes of known structure. Therefore, their derivation is dependent on the information available in limited sets of structures.

Consensus Scoring combines the information obtained from different scores to compensate for errors from individual scoring functions, therefore improving the probability of finding the correct solution. Several studies have demonstrated the success of consensus scoring methods in relation to the use of individual function schemes.

Protein Structure Modeling

Protein structures are sets of solved atomic coordinates representative of a three dimensional structure of a protein. These coordinates are solved for atoms including, but not limited to, alpha carbons, beta carbons, or side chain atoms. These sets of solved atom coordinates can also represent some substructure of a protein or polypeptide. Atom coordinates may be solved experimentally using a variety of techniques such as x-ray crystallography, electron crystallography and nuclear magnetic resonance.

Despite the accuracy of experimental techniques, they are costly and time-consuming. Advances in protein structure prediction or modeling provide methods of computationally solving the set of atom coordinates for a given protein. These methods are generally based on three different techniques (sequence comparison, threading and ab initio modeling). Protein structure prediction or modeling is usually practiced as a combination of these techniques.

A favored method in the art of protein structure prediction is to find a close homolog for which the structure is known. CASP (Critical Assessment of Techniques for Protein Structure Prediction) (Moult et al., 2003) experiments have shown that protein structure prediction methods based on homology search techniques are still the most reliable prediction methods. Sequence comparison and threading techniques are based on homology search.

Sequence comparison approaches to protein structure prediction are popular due to availability of protein sequence information. These techniques use conventional sequence search and alignment techniques such as BLAST or FASTA to assign protein fold to the query sequence based on sequence similarity.

Approaches which use protein profiles are similar to sequence-sequence comparisons. A protein profile is an n-by-20 substitution matrix where n is the number of residues for a given protein. The substitution matrix is calculated via a multiple sequence alignment of close homologs of the protein. These profiles may be searched directly against sequence or compared with each other using search and alignment techniques such as PSI-BLAST and HMMer.

It is known that sequence similarity is not necessary for structural similarity. Proteins sharing similar structure can have negligible sequence similarity. Convergent evolution can drive completely unrelated proteins to adopt the same fold. Accordingly, ‘threading’ methods of protein structure prediction were developed which use sequence to structure alignments. In threading methods, the structural environment around a residue could be translated into substitution preferences by summing the contact preferences of surrounding amino acids. Knowing the structure of a template, the contact preferences for the 20 amino acids in each position can be calculated and expressed in the form of an n-by-20 matrix. This profile has the same format as the position specific scoring profile used by sequence alignment methods, such as PSI-BLAST, and can be used to evaluate the fitness of a sequence to a structure.

Ab initio methods are aimed at finding the native structure of the protein by simulating the biological process of protein folding. These methods perform iterative conformational changes and estimate the corresponding changes in energy. Ab initio methods are complicated by inaccurate energy functions and the vast number of possible conformations a protein chain can adopt. The most successful approaches of ab initio modeling include lattice-based simulations of simplified protein models and methods building structures from fragments of proteins. Ab initio methods demand substantial computational resources and are also quite difficult to use, and expert knowledge is needed to translate the results into biologically meaningful results. Despite known limitations, Ab initio methods are increasingly applied in large-scale annotation projects, including fold assignments for small genomes. Recent examples of such applications include Bonneau et al. 2001, Kuhlman et al. 2003 and Dantas et al. 2003.

In practice, protein structure prediction typically involves a combination of the listed techniques, both experimental and computational. Hybrid approaches to protein structure prediction involve using different techniques for solving the atom coordinates at different stages or to solve for different parts of the protein structure. An example of this would be the use of AS2TS (amino acid to tertiary structure, a homology technique) to facilitate the molecular replacement (MR) phasing technique in experimental X-ray crystallographic determination of the protein structure of Mycobacterium tuberculosis (MTB) RmlC epimerase (Rv3465) from the strain H37rv. The AS2TS system was used to generate two homology models of this protein that were then successfully employed as MR targets.

Meta-predictors or consensus approaches attempt to benefit from the diversity of models by combining multiple techniques. In these methods, predictive models are collected and analyzed from a variety of different computational and experimental techniques. A common approach for combining models by consensus is to select the most abundant fold represented in the set of high scoring models. Other approaches to consensus modeling involve structural clustering such as HCPM-Hierarchical Clustering of Protein Models (Gront and Kolinski, 2005).

In one embodiment of the present invention the protein structures are predicted using the AS2TS system. The AS2TS system uses homology methods to translate sequence-structure alignment data into atom coordinates. For a given sequence of amino acids, the AS2TS (amino acid sequence to tertiary structure) system calculates (e.g. using PSI-BLAST analysis of PDB) a list of the closest proteins from the PDB, and then a set of draft 3D models is automatically created.

Near-Neighbor and Target Selection

A set of near neighbors and targets can be selected using various methods of comparison to the reference polypeptide such as sequence similarity, structural similarity, or taxonomy. Those skilled in the art can picture a variety of combinations of the following methods.

A preferred method of finding or determining near-neighbor or cutoff sets is through the use of structural similarity alignments using programs such as LGA. Using structural similarity comparison, a known protein structure may be aligned with a database of other known structures such as PDB (Protein Data Bank). Cutoff values for targets may be specified using the RMSD or distance between residues in Angstroms. Structures having good alignment but not strong enough alignment to be considered targets may be identified as nearest neighbor structures.

Sequence similarity comparisons form another method of selecting a set of near neighbors or targets. Various methods of sequence-sequence comparison (BLAST, HMMer, etc) may be used to generate a metric of sequence similarity and identify close sequence homologs or target polypeptides. Conversely, threshold values may be set to identify near neighbor polypeptides which have lower sequence similarity and are not likely to contain motifs that would co-classify with target or near neighbor proteins.

A phylogenic taxonomy provides a known or accepted classification of groups of organisms based on evolutionary relatedness. Taxonomy can be used to determine sets of near neighbors or targets. For instance, a group of related organisms may have very similar proteins to the reference protein. Depending on the application of the method, the motif may be used to distinguish a family of related organisms, which would form the set of targets. In contrast, there may be very similar organisms or viruses desirable to select against in the identification of a signature forming the set of near neighbor polypeptides. Phylogeny or taxonomy can be used to identify the largest subset of confounding pathogens or organisms, thus improving the accuracy of the method.

In the absence of a known phylogeny, a calculated molecular phylogeny may be created using sequence similarity comparisons. In these analyses, a distance matrix between similar sequences is created to generate a measure of evolutionary distance. These distances are then clustered to create phylogenetic trees representative of sequence divergence due to evolution. Common algorithms for clustering include neighbor joining and UPGMA (Unweighted Pair Group Method with Arithmetic mean) (Prager and Wilson, 1978). Phylogenetic tree data may be used to select near neighbor and target proteins in the same manner as taxonomy is used.

Given the availability and accuracy of computational protein structure prediction, those skilled in the art can easily see the benefit of combining identification of near neighbor and target polypeptides using taxonomy and sequence similarity with the preferred method of structural alignment through the creation of three dimensional models for the polypeptides. The three dimensional models may then be further evaluated by structural alignment with the reference protein in order to select near neighbor and target polypeptides.

Conservation Scores

A scoring function maps an abstract concept to a numeric value. Conservation scores are generated to assign a quantitative value to the degree of evolutionary conservation of a residue at a position in the sequence. Evolutionary conservation is defined by the phenomena in which residues at a position in a molecule are not subject to deletion or substitution in molecules within a species or homologous molecules across different species. It is inferred from conservation that the residue is integral to the function of the molecule and a substitution would cause a loss-of-function in the molecule, potentially rendering unviable the organism producing the molecule. Therefore, conservation is used as a measure of the relative functional importance of a residue.

Conservation of one or more residues is scored relative to a group of close homologs or target polypeptide sequences. These sequences may be selected by various processes as discussed in detail in the above section Near-Neighbor and Target Selection. In the scoring of conservation, various similarity metrics may be employed as discussed above in the section titled Similarity Metrics.

Scores can be calculated based on comparisons of the target and near neighbor sequence to the reference polypeptide or a consensus residue. A consensus residue can be calculated for a position in a correspondence based on the residue most frequently found in the aligned structures at that position. Scores for residues in every target sequence are generated by comparison to a corresponding reference or consensus residue, the comparison using the selected similarity metric. Scores for residues in each target sequence can then be combined into a single conservation score by averaging the score for each residue in the target sequences.

Uniqueness scores are generated to provide a numeric quantity of the uniqueness of the residue relative to a group of near neighbor polypeptides known to confound or have false or superficial similarity to the group of homologs. The uniqueness scores are used to verify the conservation scores actually represent conservation and are not spurious results. The uniqueness scores are calculated using the same methods and processes as outlined above in reference to the conservation scores.

Conservation and uniqueness scores can be combined in various ways to generate Conservation-Uniqueness scores. Combining, as referred to herein, is used to designate any mathematical operation or combination of mathematical operations including, but not limited to adding, subtracting, multiplying, or dividing. In one embodiment, the conservation scores and uniqueness scores are calculated using the same similarity metric. The uniqueness score is then subtracted from the conservation score.

Other embodiments include weighting the conservation or uniqueness scores by the number of targets or near neighbors before or during the combining of the two scores. In another embodiment of the present invention, the conservation or uniqueness scores are assigned weights to modify the stringency of the method. For instance, the uniqueness score may be assigned a lesser value than the conservation score in order to relax the stringency of the method. The use of alternate methods of weighting and normalization based on the number of sequences will be apparent to those skilled in the art.

### REFERENCES

Zhou, C E, Zemla A, Roe D, Young M, Lam M, Schoeniger J S, and Balhorn R. (2005) Computational approaches for identification of conserved/unique binding pockets in the A chain of ricin. Bioinformatics 21:3085-3096
Zhou C E, Smith J, Lam M, Zemla A, Dyer M D, Slezak T. (2007) MvirDB—a microbial database of protein toxins, virulence factors and antibiotic resistance genes for bio-defence applications. Nucleic Acids Res. January; 35
Zhou C L, Lam M W, Smith J R, Zemla A T, Dyer M D, Kuczmarski T A, Vitalis E A, Slezak T R. (2006) MannDB—a microbial database of automated protein sequence analyses and evidence integration for protein characterization. BMC Bioinformatics. October 17; 7:459.
Gardner S N, Kuczmarski T A, Zhou C E, Lam M W, Slezak T R. (2005) System to assess genome sequencing needs for viral protein diagnostics and therapeutics. J Clin Microbiol. April; 43(4):1807-17.
Kuntz, I., et. al. (1982) A Geometric Approach to Macromolecule-Ligand Interactions. J. Mol. Biol., 161, 269-288.
Altschul S F, Gish W, Miller W, Myers E W, Lipman D J (1990) Basic local alignment search tool. J Mol Biol. 1990 Oct. 5; 215(3):403-10.
McClure M A, Smith C, Elton P.(1996) Parameterization studies for the SAM and HMMER methods of hidden Markov model generation. Proc Int Conf Intell Syst Mol Biol, 4:155-64.
Pearson W R, Lipman D J. (1988) Improved tools for biological sequence comparison. Proc Natl Acad Sci USA. April; 85(8):2444-8.
Naughton B T, Fratkin E, Batzoglou S, Brutlag D L (2006) A graph-based motif detection algorithm models complex nucleotide dependencies in transcription factor binding sites. Nucleic Acids Res.; 34(20):5730-9. Epub 2006 Oct. 13.
Chen X, Jiang T. (2006) An improved Gibbs sampling method for motif discovery via sequence weighting. Comput Syst Bioinformatics Conf. 239-47.
Jensen S T, Liu J S. (2004) BioOptimizer: a Bayesian scoring function approach to motif discovery. Bioinformatics. July 10; 20(10):1557-64.
Franti P, Virmajoki O, Hautamaki V. (2006) Fast agglomerative clustering using a k-nearest neighbor graph. IEEE Trans Pattern Anal Mach Intell. 2006 November; 28(11):1875-81.
Bottegoni G, Cavalli A, Recanatini M. (2006) A comparative study on the application of hierarchical-agglomerative clustering approaches to organize outputs of reiterated docking runs. J Chem Inf Model. March-April; 46(2):852-62.
Sousa S F, Fernandes P A, Ramos M J. (2006) Protein-ligand docking: current status and future challenges. Proteins. October 1; 65(1):15-26.
Rost, B., Liu, J. (2005) The PredictProtein server. Nucleic Acids Res. 2003 Jul. 1; 31(13):3300-4.
Gront D., Kolinski A., HCPM—program for hierarchical clustering of protein models. Bioinformatics. July 15; 21(14):3179-80. Epub 2005 Apr. 19.
Moult, J., Fidelis, K., Zemla, A. (2003) Hubbard T., Critical assessment of methods of protein structure prediction (CASP)-round V., Proteins.; 53 Suppl 6:334-9.
Prager, E. M., Wilson, A. C. (1978) Construction of phylogenetic trees for proteins and nucleic acids: empirical evaluation of alternative matrix methods. J Mol Evol. June 20; 11(2):129-42.
Bonneau, R., Tsai, J., Ruczinski, I. and Baker, D. (2001) Functional inferences from blind ab initio protein structure predictions. J. Struct. Biol., 134, 186-190.
Kuhlman, B., Dantas, G., Ireton, G. C., Varani, G., Stoddard, B. L. and Baker, D. (2003) Design of a novel globular protein fold with atomic-level accuracy. Science, 302, 1364-1368. 61.
Dantas, G., Kuhlman, B., Callender, D., Wong, M. and Baker, D. (2003) A large scale test of computational protein design: folding and stability of nine completely redesigned globular proteins. J. Mol. Biol., 332, 449-460.
Attwood, T. K., Avison, H., Beck, M. E., Bewley, M., Bleasby, A. J., Brewster, F., Cooper, P., Degtyarendko, K., Geddes, A. J., Flower, D. R., Kelly, M. P., Lott, S., Measures, K. M., Parry-Smith, D. J., Perkins, D. N., Scordis, P., Scott, D., and Worledge, C. (1997) The PRINTS database of protein fingerprints: A novel information resource for computational molecular biology. J Chem Inf Comput Sci, 37, 417-424.
Berman, H. M., Westbrook, J., Feng, Z., Gilliland, G., Bhat, T. N., Weissig, H., Shindyalov, I. N., and Bourne, P. E. (2000) The protein data bank. Nucleic Acids Research, 8, 235-242.
Bower, M. J., Cohen, F. E. and Dunbrack, R. L. (1997) Prediction of protein side-chain rotamers from a backbone-dependent rotamer library: a new homology modeling tool. J Mol Biol, 267, 1268-1282.
Canutescu A. A., Shelenkov A. A. and Dunbrack, R. L. (2003) A graph theory algorithm for protein side-chain prediction. Prot Sci, 12, 2001-2014.
Day, P. J., Ernst, S. R., Frankel, A. E., Monzingo, A. F., Pascal, J. M., Molina-Svinth, M. C. and Robertus, J. D. (1996) Structure and activity of an active site substitution of ricin A chain. Biochemistry, 35, 11098-11103.
Ewing, T. J. A., S. Makino, A. G. Skillman, I. D. Kuntz. 2001. DOCK 4.0: Search strategies for automated molecular docking of flexible molecule databases. Journal of Computer-Aided Molecular Design 15: 411-428.
Fygenson, D. K., Needlemen, D. J. and Sneppen, K. (2004) Variability-based sequence alignment identifies residues responsible for functional differences in a and b tubulin. Protein Science, 13, 25-31.
Gabdoulkhakov, A. G., Savochkina, Y., Konareva, N., Krauspenhaar, R., Stoeva, S., Nikonov, S. V., Voelter, W., Betzel, C., Mickhailov, A. M. Structure-Function Investigation Comlex of Agglutinin from Ricinus Communis with Galactoaza (to be published).
Gardner, S., Lam, M. W., Mulakken, N. J., Torres, C. L., Smith, J. R. and Slezak, T. R. (2004) Sequencing needs for viral diagnostics. Journal of Clinical Microbiology, 42, 0095-1137.
Hubbard, S. J. and Thornton, J. M. (1993) ‘NACCESS’, Computer Program, Department of Biochemistry and Molecular Biology, University College, London. Karlin, S. and Ghandour, G. (1985) Multiple-alphabet amino acid sequence comparison of the immunoglobulin k-chain constant domain. Proc. Natl. Acad. Sci. USA, 82, 8597-8601.
Knight, B. (1979) Ricin—a potent homicidal poison. British Medical Journal, 278, 350-351.
Kuntz, I. D., Blaney, J. M., Oatley, S. J., Langridge, R. and Ferrin, T. E. (1982) A geometric approach to macromolecule-ligand interactions. J. Mol. Biol., 161, 269-288.
Lebeda, F. J. and Olson, M. A. (1999) Prediction of a conserved, neutralizing epitope in ribosome-inactivating proteins. International Journal of Biological Macromolecules, 24, 19-26.
Lightstone, F. C., Prieto, M. C., Singh, A. K., Piqueras, M. C., Whittal, R. M., Knapp, M. S., Balhorn, R. and Roe, D. C. (2000) Identification of novel small molecule ligands that bind to tetanus toxin. Chem Res Toxicol., 13, 356-362.
Lord, J. M., Roberts, L. M. and Robertus, J. D. (1994) Ricin: structure, mode of action, and some current applications. FASEB J, 8, 201-208.
Marsden, C. J., Fulop, V., Day, P. J and Lord, J. M. (2004) The effects of mutations surrounding and within the active site on the catalytic activity of ricin A chain. Eur. J. Biochem., 271, 153-162. 12
Olson, M. A., Carra, J. H., Roxas-Duncan, V., Wannemacher, R. W., Smith, L. A., and Millard, C. B. (2004) Finding a new vaccine in the ricin protein fold. Protein Engineering, Design & Selection, 17, 391-397.
Olsnes, S. and Kozlov, J. V. (2001) Ricin. Toxicon 39:1723-1728.
Ouzounis, C. A., Coulson, R. M., Enright, A. J., Kunin, V., Pereira-Leal, J. B. (2003) Classification schemes for protein structure and function. Nat Rev Genet., 4, 508-519.
Peruski, A. H., and Peruski, Jr, L. F. (2003) Immunological methods for detection and identification of infectious disease and biological warfare agents. Clinical and Diagnostic Laboratory Immunology, 10, 506-513.
Portefaix, J.-M., S. Thebault, F. Bourgain-Guglielmetti, M. D. Del Rio, C. Granier, J.-C. Mani, I. Navarro-Teulon, M. Nicolas, T. Soussi, and B. Pau. 2000. Critical residues of epitopes recognized by several anti-p53 monoclonal antibodies correspond to key residues of p53 involved in interactions with the mdm2 protein. Journal of Immunological methods 244: 17-28.
Sayle, R. A. and Milner-White, E. J. 1995. RasMol: Biomolecular graphics for all. Trends in Biochemical Sciences, 20, 374-376.
Shuker, S. B., Hajduk, P. J., Meadows, R. P. and Fesik, S. W. (1996) Discovering High-Affinity Ligands for Proteins: SAR by NMR. Science, 274, 1531-1534.
Slezak, T., Kuczmarski, T., Ott, L., Torres, C., Medeiros, D., Smith, J., Truitt, B., Mulakken, N., Lam, M., Vitalis, E., Zemla, A., Zhou, C. E. and Gardner, S. (2003) Comparative genomics tools applied to bioterrorism defense. Briefings in Bioinformatics, 4, 133-149.
Wang, G., De, J., Schoeniger, J. S., Roe, D. C. and Carbonell, R. G. (2004) A hexamer peptide ligand that binds selectively to staphylococcal enterotoxin B: isolation from a solid phase combinatorial library. Journal of Peptide Research, 64, 51-64.
Wesche, J., Rapak, A. and Olsnes, S. (1999) Dependence of ricin toxicity on translocation of the toxin A-chain from the endoplasmic reticulum to the cytosol. J Biol Chem, 274, 34443-34449.
Weston, S. A., Tucker, A. D., Thatcher, D. R., Derbyshire, D. J. and Pauptit, R. A. (1994) X-ray structure of recombinant ricin A-chain at 1.8 Å resolution. J Mol Biol., 244, 410-422.
Yan, X., Hollis, T., Svinth, M., Day, P., Monzingo, A. F., Milne, G. W., Robertus, J. D. (1997) Structure-based identification of a ricin inhibitor. J Mol Biol, 266, 1043.
Zemla, A. (2003) LGA: a method for finding 3D similarities in protein structures. Nucleic Acid Research, 31, 3370-3374.
Zemla, A., Ecale Zhou, C., Slezak, T., Kuczmarski, T., Rama, D., Torres, C, Sawicka, D. and Barsky, D. (2005) AS2TS system for protein structure modeling and analysis. Nucleic Acids Research, 1; 33 (Web Server issue): W111-5.
Higgins D., Thompson J., Gibson T., Thompson J. D., Higgins D. G., Gibson T. J. (1994) CLUSTAL W: improving the sensitivity of progressive multiple sequence alignment through sequence weighting, position-specific gap penalties and weight matrix choice. Nucleic Acids Res. 22:4673-4680.
Schwartz A. S., and Pachter L. (2007) Multiple Alignment by Sequence Annealing. Bioinformatics, 23 e24-e29.
Petryszak R, Kretschmann E, Wieser D, and Apweiler R (2005) The predictive power of the CluSTr database. Bioinformatics. 2005 Sep. 15; 21(18):3604-9
Chen Y, Reilly K D, Sprague A P, and Guan Z (2006) SEQOPTICS: A protein sequence clustering system. BMC Bioinformatics, 7:210.
Henikoff S and Henikoff J G. (1992) Amino acid substitution matrices from protein blocks. PNAS, 89:10915-9.
Wishart D S, Knox C, Guo A C, Shrivastava S, Hassanali M, Stothard P, Chang Z, Woolsey J (2006) DrugBank: a comprehensive resource for in silico drug discovery and exploration. Nucleic Acids Research, 1:34.
Schomburg I., Chang A., Ebeling C., Gremse M., Heldt C., Huhn G., Schomburg D. (2004) BRENDA, the enzyme database: updates and major new developments. Nucleic Acids Res. 2004 Jan. 1; 32 Database issue: D431-3.
Faulon, Jean-Loup, Collins M. J., Carr R. D. (2004) The Signature Molecular Descriptor. 4. Canonizing Molecules Using Extended Valence Sequences. Journal of Chemical Information and Modeling 44(2): 427-436
Willett, Peter. (2005) Molecular similarity approaches for chemoinformatics. 229^{th} ACS National Meeting, March 13-17, San Diego, Calif.
McMartin C and Bohacek R S (2004) Flexible matching of test ligands to a 3D pharmacophore using a molecular superposition force field: comparison of predicted and experimental conformations of inhibitors of three enzymes. Journal of Computer-Aided Molecular Design, 9:237-250.
Waszkowycz B, Perkins T D J, Sykes R A, and Li J (2001) Large-scale virtual screening for discovering leads in the postgenomic ear. IBM Systems Journal, vol. 40.
Bayley M J, Gillet V J, Willett P, Bradshaw J, Green D V S (1999) Computational analysis of molecular diversity for drug discovery. Proceedings of the Third Annual International Conference on Computational Molecular Biology, Lyon, France, pp. 321-330.
Pearl F, Todd A, Sillitoe I, Dibley M, Redfern O, Lewis T, Bennett C, Marsden R, Grant A, Lee D, Akpor A, Maibaum M, Harrison A, Dallman T, Reeves G, Diboun I, Addou S, Lise S, Johnston C, Sillero A, Thornton J, Orengo C., (2005) The CATH Domain Structure Database and related resources Gene3D and DHS provide comprehensive domain family information for genome analysis. Nucleic Acids Research. Vol. 33 Database Issue D247-D251
Chen B Y, Fofanov V Y, Kristensen D M, Kimmel M, Lichtarge O, and Kavriaki L E (2005) Algorithms for structural comparison and statistical analysis of 3D protein motifs. Pacific Symposium on Biocomputing, 10: 334-345.
Heller K A, and Ghahramani Z: Bayesian Hierarchical Clustering. in ACM International Conference Proceeding Series, 2005. 119:297-304. ISBN: 1-59593-180-5. ACM Press.
Liang J, Edelsbrunner H, Fu P, Sudhakar P V, Subramaniam S. (1998) Analytical shape computation of macromolecules. II. Identification and computation of inaccessible cavities in proteins. Proteins: Struct. Funct. Genet. 33:18-29.
Laurie A T, Jackson R M (2005) Q-SiteFinder: an energy-based method for the prediction of protein-ligand binding sites. Bioinformatics, 21: 1908-1916.
Miller M D, Kearsley S K, Underwood D J, and Sheridan R P (1994) FLOG: a system to select ‘quasi-flexible’ ligands complementary to a receptor of known three-dimensional structure. Journal of Computer-Aided Molecular Design, 8:153-174.
Glover F W, and Laguna M (1998) Tabu Search. Kluwer Academic Publishers.
Morales L B, Garduno-Juarez R, Aguilar-Alvarado J M, and Riveros-Castro F J (2000) A parallel tabu search for conformational energy optimization of oligopeptides. Journal of Computational Chemistry, 21:147-156.

What is claimed is:

1. A method of identifying a set of target pockets for broad-spectrum drug development, wherein each target pocket comprises a three-dimensional concavity in a protein structure, the method comprising:
providing a set of protein motifs, wherein each protein motif of the set of motifs comprises a first plurality of conserved residues;
identifying a plurality of pockets based on the set of protein motifs, wherein each pocket comprises a second plurality of conserved residues that define the three dimensional concavity on the surface of a protein structure, wherein the second plurality of conserved residues correspond at least in part with the first plurality of conserved residues;
generating a plurality of binding profiles in association with the plurality of pockets, wherein each binding profile specifies at least one calculated binding activity between each pocket and at least one test molecule;
generating a plurality of pocket similarity values based on the plurality of binding profiles, wherein each pocket similarity value is based on binding profiles associated with at least two pockets of the plurality of pockets;
identifying a set of target pockets based on the plurality of pocket similarity values; and
storing the set of target pockets.

2. The method of claim 1, wherein identifying a set of target pockets based on the plurality of pocket similarity values comprises:
generating a cluster based on the plurality of pocket similarity values; and
selecting a set of target pockets based on the cluster.

3. The method of claim 1, wherein the binding profiles further specify a plurality of calculated binding activities between each pocket and a plurality of test molecules.

4. The method of claim 1, wherein the second plurality of conserved residues is less than 4 residues.

5. The method of claim 1, wherein each pocket similarity value is based on each binding profile associated with each pocket and a representative binding profile associated with a representative pocket.

6. The method of claim 3, further comprising:
generating a plurality of molecule similarity values based on the plurality of binding profiles, wherein each molecule similarity value is based on binding profiles associated with at least two pockets of the plurality of pockets; and
identifying a set of target molecules based on the plurality of molecule similarity values.

7. The method of claim 1, wherein the first plurality of conserved residues are conserved in a set of three-dimensional protein structures.

8. The method of claim 1, wherein the first plurality of conserved residues are conserved in a set of protein sequences.

9. The method of claim 1, wherein providing a set of protein motifs further comprises:
identifying a plurality of protein motifs, wherein each protein motif of the plurality of protein motifs comprises a plurality of conserved residues and is associated with a protein sequence and protein structure;
generating a plurality of protein motif similarity values, wherein each protein motif similarity value is based on the sequence of a reference protein comprising the protein motif, the structure of the reference protein comprising the protein motif or any combination thereof; and
identifying a set of protein motifs based on the plurality of protein motif similarity values.

10. The method of claim 9, further comprising identifying the plurality of protein motifs, wherein identifying the plurality of protein motifs comprises:
providing a plurality of sets of aligned three-dimensional protein structures;
identifying a plurality of spans from the plurality of sets of aligned three-dimensional protein structures, wherein each span is comprised of a plurality of residue positions and each residue position is comprised of a one-to-one set of corresponding residues from the aligned three-dimensional structures whose positions differ by less than a pre-determined distance;
generating a plurality of conservation scores for a plurality of residue positions in the span, wherein each conservation score is generated based on a similarity metric and a one-to-one set of corresponding residues; and
identifying a protein plurality of motifs, wherein each motif is based on the generated plurality of conservation scores.

11. The method of claim 10, wherein the pre-determined distance is less than 5 Angstroms.

12. The method of claim 10, further comprising generating the plurality of sets of aligned three-dimensional structures wherein generating each set of aligned three dimensional structures comprises
identifying a set of homologous three-dimensional protein structures; and
determining the set of aligned three-dimensional protein structures based on a local alignment of the homologous three-dimensional protein structures, a global alignment of the homologous three-dimensional protein structures or any combination thereof.

13. The method of claim 12, wherein the set of homologous three-dimensional structures comprises a structure obtained using x-ray crystallography, electron crystallography, nuclear magnetic resonance, computational protein structure modeling, or combinations thereof.

14. The method of claim 1, wherein identifying a plurality of pockets based on the set of motifs further comprises:
generating a set of three-dimensional spheres associated with co-ordinates in three-dimensional space, wherein the set of three-dimensional spheres represent a negative image of the surface of the protein structure;
determining a subset of the set of spheres that fall within a second pre-determined distance from the second set of conserved residues in the protein structure based on the co-ordinates of the set of spheres and the co-ordinates of the second set of conserved residues in the protein structure; and
determining that the second set of conserved residues form a three-dimensional concavity on the surface of the protein structure based on the co-ordinates of the subset of the set of spheres.

15. The method of claim 14, wherein the second pre-determined distance is less than 8 Angstroms.

16. The method of claim 1, further comprising generating the calculated binding activity between each pocket and each test molecule based on generating a docking between the test molecule and the pocket based on computational protein-ligand docking.

17. The method of claim 9, further comprising identifying the plurality of protein motifs, wherein identifying the plurality of protein motifs comprises:
providing a set protein sequences;
generating a sequence alignment of the protein sequences based on a multiple sequence alignment, a pair-wise sequence alignment or any combination thereof;
identifying a plurality of conserved residues in each protein sequence based at least in part on the sequence alignment; and
identifying a plurality of protein motifs, wherein each protein motif comprises the plurality of conserved residues in each protein sequence.

18. The method of claim 17, further comprising:
identifying a plurality of test molecules based on the motifs;
determining a plurality of quantitative structural relationships, wherein each quantitative structural relationships is based on the binding activity between the set of motifs and the plurality of test molecules.

19. A computer-readable storage medium comprising computer program code for identifying a set of target pockets for broad-spectrum drug development, wherein each target pocket comprises a three-dimensional concavity in a protein structure, the computer program code for:
providing a set of protein motifs, wherein each protein motif of the set of motifs comprises a first plurality of conserved residues;
identifying a plurality of pockets based on the set of protein motifs, wherein each pocket comprises a second plurality of conserved residues form a three dimensional concavity on the surface of a protein structure, wherein the second plurality of conserved residues correspond at least in part with the first plurality of conserved residues;
generating a plurality of binding profiles in association with the plurality of pockets, wherein each binding profile specifies at least one calculated binding activity between each pocket and at least one test molecule;
generating a plurality of pocket similarity values based on the plurality of binding profiles, wherein each pocket similarity value is based on binding profiles associated with at least two pockets of the plurality of pockets;
identifying a set of target pockets based on the plurality of pocket similarity values; and
storing the set of target pockets.

20. The computer-readable storage medium of claim 19, wherein providing a set of motifs further comprises:
identifying a plurality of protein motifs, wherein each protein motif of the plurality of protein motifs comprises a plurality of conserved residues and is associated with a protein sequence and protein structure;
generating a plurality of protein motif similarity values, wherein each protein motif similarity value is based on the sequence of a reference protein comprising the protein motif, the structure of the reference protein comprising the protein motif or any combination thereof; and
identifying a set of protein motifs based on the plurality of protein motif similarity values.

21. The computer-readable storage medium of claim 19, further comprising generating the plurality of motifs, wherein generating the plurality of motifs comprises:
providing a plurality of sets of aligned three-dimensional protein structures;
identifying a plurality of spans from the plurality of sets of aligned three-dimensional protein structures, wherein each span is comprised of a plurality of residue positions and each residue position is comprised of a one-to-one set of corresponding residues from the aligned three-dimensional structures whose positions differ by less than a pre-determined distance;
generating a plurality of conservation scores for a plurality of residue positions in the span, wherein each conservation score is generated based on a similarity metric and a one-to-one set of corresponding residues; and
identifying a protein plurality of motifs, wherein each motif is based on the generated plurality of conservation scores.
